(19) United States
(12) Patent Application Publication
(10) Pub. No.: US 2007/0105120 A1
(43) Pub. Date: May 10, 2007

(54) Method of identifying/designing and/or modifying chemical species capable of interacting with a physiologically active macromolecule

(76) Inventor: Burra V.L.S. Prasad, Hyderabad (IN)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/579,171

(22) PCT Filed: Jun. 18, 2003
(86) PCT No.: PCT/IB03/03444

§ 371 Date: Jan. 22, 2007

(51) Int. Cl. G06F 19/00; C12Q 1/68

(52) U.S. Cl. 435/6; 702/20

(57) ABSTRACT

The present invention relates to a method of identifying/designing and/or modifying species capable of interacting with a protein molecule that is a potential drug target.

The present invention relates to a method of identifying/designing and/or modifying species capable of interacting with a protein molecule that is a potential drug target.

Despite the availability of voluminous literature and huge structural knowledge base on bio-macromolecules in general and proteins in particular, a) it is still unclear, in its entirety, how a protein attains its native architecture essential for its predefined function b) therefore, rational design of proteins, peptides or drugs is not yet possible, and c) the so-called “protein folding problem”, i.e. the question of how the three-dimensional structure of a protein is determined by its primary amino acid sequence, persists. Consequently, the long standing desire of biologists to reach the stage of designing a “customized protein: a step towards nano-biotechnology revolution” for social benefits, is still an untealised dream.

Historically, as more protein structures began to be solved it slowly became clear that the seemingly random arrays of secondary structural motifs connected by loop regions can, in fact, be categorized and classified into groups that share the same or similar fold or have similar folding motifs (Chothia, 1984 Annu. Rev. Biochem. 53:537-572; Finkelstein and Ptitsyn, 1987, Prog. Biophys. Mol. Biol. 50:171-190; Chothia and Finkelstein, 1990, Annu. Rev. Biochem. 59:1007-1039). A classification of structures based on their common structural patents and folding motifs is the “Structural Classification of Proteins” data base (SCOP) (Murzin, Brenner et al., 1995, Journal of Molecular Biology 247(4): 536-540) which is a reflection of the fact that there is indeed some regularity within the structural diversity and variability of proteins.

From inspection and analysis of the three-dimensional structures various semi-empirical rules on the way that proteins obtain their native three-dimensional confirmation based on their primary structure, could be established (reviewed in (Chothia and Finkelstein, 1990, ibid.)). Most of these rules have initially been derived from analysis and description of the structures and have later then been rationalized using biophysical, statistical or geometrical arguments. Whilst a comprehensive list of these rules is beyond the limits of this application, a view points are worth mentioning:

The most important determinants of folding patterns are the secondary structural elements of a protein and the hydrophobic surfaces they form. The arrangement of the secondary structural elements is most likely to be determined by a number of factors (Chothia and Finkelstein, 1990, ibid):

Firstly, intermediates on the folding pathway may have a preference for certain chain topologies over others, because these topologies are either lower in energy or kinetically more accessible, and this will result in a certain assembly of helices and sheets. Secondly the arrangement of the polypeptide chain has to occur in such a manner that an extremely close packing for the majority of buried residues is achieved (Karpusas, Baase et al., 1989, Proc. Natl. Acad. Sci. USA 86:8237-8241). Only few arrangements will satisfy this constraint and the native confirmation is the most ideal one.

Thirdly the burial of surfaces has to provide enough stability for a specific (i.e. the native) assembly of secondary structural elements to be feasible. Extensive mutational studies show that both introduction (Karpusas, Baase et al., 1989, ibid) or removal (Kellis, Nyberg et al., 1989, Biochemistry 28:4914-4922) of individual methyl or methylene groups from the protein interior destabilise the native structure, showing that in naturally occurring proteins the burial of hydrophobic surfaces is very much ideal for the given native confirmation, and emphasizing the validity, at least in many cases, of Anfinsen's original thermodynamic hypothesis (Anfinsen, 1973, Science 181:223-230) on the native confirmation being the most stable one.

In addition to these fundamental rules, a whole battery of other “folding instructions” regarding the modalities and details of packing arrangements have been established:

For a protein to be compact and to satisfy the hydrogen bonding potential of buried polar groups, it has to form extensive secondary structure over wide stretches of the polypeptide chain. Whilst α-helices and/or β-strands establish an extensive intramolecular hydrogen-bonding network, and hence can form part both of the core and of the surface regions within the molecule, the connecting loop regions establish very few intramolecular hydrogen-bonds and therefore are structural elements that can nearly always be found on the surface where they only hydrogen bond to water.

For the tight packing of secondary structural elements together and the topology, i.e. the path that the polypeptide chain follows through the molecule, there are certain restrictions imposed by the rigidity of the polypeptide chain. For example it has been argued that the bending of a loop of 10 residues by 180° is not as costly in free energy terms (3.5 kcal/mol) as bending it by 360° would be (6 kcal/mol) (Finkelstein and Ptitsyn, 1987, ibid). As a consequence, secondary structural elements that are linked by a loop and packed together are more often aligned in an antiparallel fashion (180° loop) than a parallel fashion (360°). It is not clear, however, that these arguments which are derived from polymer physics and applied to flexible structures, are valid in a thermodynamic analysis of folding to an essentially rigid structure. They may, however, be of more relevance to partially folded states and hence give rise to structural preferences through the folding pathway. Likewise, the rigidity of the chain causes β-x-β units to prefer a right-handedness in their topology, because the bending angle in these units is greater and hence, energetically more unfavourable, for left-handed connections than for right-handed ones, due to the right-handed twist found in most β-strands.

Moving from super-secondary structural motifs to the special arrangement and topology of helices within a single globular domain, it has been found that the packing together of α-helices can be described by a simple geometrical model, the so-called “quasispherical polyhedron model” (Murzin and Finkelstein, 1988, J. Mol. Biol. 204:749-770) according to which the helices pack together in such a way that they form the edges of an imaginary polyhedron encompassing a central hydrophobic core with a diameter of about 11 Å. The arrangement of helices is as spherical as possible. The packing together of three helices is ideal in an octahedron, four helices are packed ideally in a dodecahedron, five helices in a hexadecahedron and six helices in an icosahedron. A comparison of solved protein'structures with these ideal models confirmed the validity of the approach, whilst also showing that, for example of the ten different possible ways of arranging four helices in their dodecahedron, only one arrangement is favoured in nature, in which neighboring helices are inclined at angles of −50° and/or +20°. These are values expected also from general characteristics of helix surfaces (Chothia, Levitt et al., 1981, J. Mol. Biol. 145:215-250) where side-chains form grooves and ridges. The packing together of two helices along these grooves and ridges will be ideal when the helices are inclined at −50° or +20° to each other. Deviations from the ideal polyhedron model are observed in nature when the helices gather together in a non-spherical rather elongated fashion (Murzin and Finkelstein, 1988, ibid). Arrangements of more than six helices in one domain cannot be fitted to a polyhedron and hence have to adopt alternatives like, for example, forming layered structures.

It is clear from the above, that the detection of common folding patterns and motifs and their classification appears to be, methodologically speaking, a rather phenomenological approach, that, so far, has not yielded a complete answer to the problem of the sequence-structure relation ship of proteins. This methodology has the great merit of putting some order into the initial random appearance of solved protein structures and being able to generally rationalize retrospectively why certain folding patterns are preferred over others. Yet, it is not accurate enough, to actually predict a specific folding pattern for a specific sequence.

The picture becomes more complicated, when it comes to the interaction of small molecules that are suspected of interacting with a protein molecule that is a potential drug target. These small molecule drug candidates need to be built into the protein structure by molecular modelling approaches, and from their in-silico-behavior, in some cases, conclusions can be made with respect to their potential as a drug, acting as an inhibitor or allosteric modulator of the protein. The present day approaches to molecular recognition are still predictive and not design oriented which always leave a result to be probabilistic than deterministic and hence not-absolutely-reliable.

It becomes even more complex to comprehend and attempt to predict and/or design macro-molecule-macromolecule interactions than predicting a small molecule drug interaction with target. The number of atomic and molecular parameters to be analysed and calculated will be enormously large when compared to a small molecule. All the predictive modeling approaches are hampered by the tremendous amount of computer time and/or storage space required. Furthermore some times they are too rigid and schematic leading to irrelevant results.

As early as 1981, K. Eric Drexler has argued that the predictive approach adopted by natural scientist, as against the engineer's approach, has major limitation to realize the dream of creating artificial nanomachines systems (K. Eric Drexler, “Molecular Engineering, A general Approach, 1981, PNAS). He compared and discussed basic mechanical components for constructing a machine systems and their biomolecular equivalents. He proposed that “the engineering problem of designing proteins to fold in a predetermined way is must easier than the scientific problem of predicting how natural proteins fold”. Hence, we are still unable to clearly pin down the “design principles” and hence it is the bottle neck for progress to realize the dream of a “customized macromolecule”. The alternative is to adopt the engineer's approach of designing, where the designer need not seek to understand all proteins but only enough to produce useful systems in a reasonable number of attempts. Can the macromolecular design principles be realized through any engineering methodology?

The recent discoveries and successful elucidation of natural nanomachine systems such as T4 bacteriophage structure and large biomacromoleculear assemblages such as ribosomes, ATPase rotary motor, bacterial flagellar motors and actin-myosin translation system provide a ray of hope that protein designing principles can be realized. These discoveries suggest that the architecture of the cell and the macromolecules has a more important role to play in the coordination, cooperation, assembling and functioning than their chemical composition (David. E. Ingber, The Architecture of Life; January 1998; Scientific American).

It is relatively easy to identify components of complex biomolecular assemblages as has been realized in the above said examples. However, does such a component system exist with in a single macromolecule? Is a macromolecule a biological IC (Irreducible complex) (Michael Behe, “Darwin's Black Box”, Free cell Press, 1996) or can a single macromolecule be further divided into different architectural components, despite of its continuous chain? So far, none of the aforementioned “engineering” approaches has given a systematic methodology to analyse macromolecular structures and identify possible interactions between macromolecules.

Accordingly, it has been an objective of the present invention to provide for a practical method yielding more and systematic insight into macromolecular structures and allowing for the identification/modification/design/optimization of species potentially interacting with macromolecules.

It has also been an object of the present invention to provide for a method allowing a systematic and numerically based analysis of macromolecular structures. It has furthermore been an object of the present invention to provide for a method for the aforementioned purposes that is easy to perform and can be used in a very versatile manner. It has also been an object of the present invention to provide a method which enables the use of machine design principles and tensegrity principles by which the understanding of biological complexity may be further rationalised.

All these objects are solved by a method of identifying and/or designing and/or modifying and/or optimising species capable of interacting with a macromolecule, comprising the steps:
a) defining a set of physiological functions and/or properties of said macromolecule and/or said species, said physiological functions and/or properties being based on empirical data, available for said macromolecule and/or said species,
b) identifying a mechanical analogue from the set of physiological functions and/or properties of said macromolecule and/or said species, which mechanical analogue performs a mechanical function that is analogous to the physiological function of said macromolecule and/or species, and which mechanical analogue performs the mechanical function as a whole or which mechanical analogue consists of parts allowing said mechanical analogue to perform its mechanical function, such that each of said parts of said mechanical analogue performs a component of said mechanical function,
c) providing at least one structure of said macromolecule and/or species, said structure being a representation of the arrangement and connectivity of the atoms of said macromolecule and/or said species, in three dimensional space, or said structure being a set of all coordinates of the atoms of said macromolecule and/or species in three dimensional space, said macromolecule and/or species consisting of building blocks, referred to as residues,
d) identifying a group of residues within the structure of said macromolecule and/or species, said group of residues performing a specific component of physiological function of said macromolecule and/or species, or said group of residues performing a part of said physiological function of said macromolecule and/or species, which group of residues are analogous to a part of said mechanical analogue (identified in b) which performs a part of said mechanical function, said group of residues being referred to as a macromolecule architectural component (MAC), said component of physiological function of said macromolecule having its counterpart in at least one part of the mechanical analogue, identified in b), which mechanical analogue part performs an analogous component of mechanical function in said mechanical analogue,
e) repeating step d) as many times as necessary until all macromolecule architectural components are identified which are necessary for said macromolecule and/or species to perform its physiological function,
f) representing each MAC identified in step d) by a geometrical shape, which shape approximates the dimensions of said MAC,
g) assigning the approximate dimensions to each geometrical shape of step f), thereby defining the coordinates and dimensions of each MAC,
h) calculating the centres of mass and inter-MAC angles using the coordinates of each MAC,
i) parameterising the identification/design/modification/optimization of species capable of interacting with said macromolecule, by using the inter MAC-angles, centres of mass and the dimensions of the MACs.

Preferably, the method further comprises the steps:
j) physically providing/designing/modifying/optimizing a species suspected of interacting with said macromolecule, the identity of said species being based on information retrieved from performing steps a)-i) on said species as well as on said macromolecule,
k) physically providing said macromolecule,
l) physically mixing said species and said macromolecule and measuring an interaction.

In one embodiment the order of steps j) and k) is reverse.

It is preferred that said macromolecule is selected from the group comprising proteins, nucleic acids, carbohydrates, lipids and fats wherein, preferably, said macromolecule is selected from the group comprising A-DNA, B-DNA, Z-DNA, RNA, in particular t-RNA, r-RNA and m-RNA, ribozymes, proteins, protein complexes, peptides, peptidoglycans, carbohydrates, lipids and fats.

In one embodiment said species is selected from the group comprising proteins, peptides, nucleic acids, carbohydrates, lipids, fats, non-protein co-factors, small-molecule-compounds, radicals, ions and macromelecule associated water molecules, wherein, preferably, said small-molecule-compounds have a molecular mass in the range of 150-1300, preferably 200-900, more preferably 300-600.

In one embodiment the approximate dimensions assigned in step g) are in Ångstroms.

In one embodiment said macromolecule is pictorially represented using inter-MAC angles, centres of mass of said MACs and said dimensions of said MACs.

In a preferred embodiment, the pictorial representation is by means of the geometrical shapes identified in step f), whereby each MAC is represented independently by a geometrical shape, such that the geometrical shape of one MAC may be the same as that of another MAC or they may be different.

In one embodiment in steps d) to f) the MACs are construed by referring to said structure or to said set of coordinates provided in step c), to assign a geometric shape and dimensions, based on said empirical data.

It is preferred that the macromolecule architectural component identified in d) comprises residues which are more than 2 Å apart.

Preferably, the macromolecule architectural component occurs within a part of the tertiary structure of the macromolecule that is well defined, as judged by X-ray-data and/or NMR-data and/or homology modelling studies wherein, preferably, said macromolecule is a protein, and wherein, more preferably, the macromolecule architectural component occurs within a part of the tertiary structure of the protein, the Cα-atoms of which have B-factors in the range of from 2 Å²-200 Å².

In one embodiment the macromolecule architectural component occurs in a region of the tertiary structure of the macromolecule the backbone atoms of which have a root mean square deviation (RMSD) in the range of from 0,05 Å-4,0 Å, preferably in the range of from 0.4 Å-1.2 Å.

Preferably said macromolecule architectural component(s) is (are independently) represented by a geometrical shape, said shape being selected from the group comprising planes, parallelepipeds, cubes, cylinders, spirals, rings, tori, ellipsoids, balls and any combination thereof, wherein, more preferably, said geometrical shape represents/is similar to a mechanical part of a machine, such as planks/sheets, springs, tubes, screws, bolts, nuts, rivets, bushings, bearings and other components used for manufacturing a machine or component of a machine.

In one embodiment the geometrical shape selected for a MAC is a plane, wherein, preferably, a macromolecule architectural component is represented by a difference vector matrix A, wherein
‘A’=[(xᵢ−<x>) (yᵢ−<y>) (zᵢ−<z>)]
(xᵢ, yᵢ, zᵢ)=[X, Y, Z] coordinates of the central atom of each residue in the MAC, e.g. of the Cα atom, in the PAC
(<x>, <y>, <z>)=(Σxᵢ/n, Σyᵢ/n, Σzᵢ/n)
n=Number of central atoms of each residue in each MAC, e.g. of Cα atoms in each PAC.

More preferably, said difference vector matrix A is solved to yield a singular vector which represents the direction cosine of the vector which is normal to the best-fitting plane of the given coordinates of the macromolecule architectural component.

In one embodiment the geometrical shape selected for a MAC is a cylinder or a spiral, wherein, preferably, a directional vector intersecting with the longitudinal axis of said cylinder or spiral is calculated.

More preferably, said calculation occurs by the method of bisection of vectors.

In one embodiment the physiological functions and/or properties of the macromolecule are selected from the group comprising oxidoreductase, transferase, hydrolase, lyase, isomerase and ligase, wherein, preferably, the physiological functions and/or properties of the macromolecule are selected from the group comprising protease, kinase, phosphorylase, DNAase, RNAase, lipase and polymerase.

In one embodiment the physiological functions and/or properties of the macromolecule are selected from the group comprising regulatory function in cell metabolism, regulatory function in transcription and/or translation, regulatory function in signal transduction pathways, structural function, storage function, motility function, transport function, and recognition function.

In one embodiment the measurement of an interaction between the species suspected of interacting with said macromolecule and said macromolecule, in step l), occurs by UV-vis-absorption spectroscopy, fluorescence spectroscopy, circular dichroism, NMR-spectroscopy, surface plasmon resonance spectroscopy, gelfiltration, ultracentrifugation, viscometry, electrophoresis, and/or any combination of the aforementioned techniques.

The objects of the present invention are also solved by a species and/or macromolecule identified/designed/modified/optimized by the method according to the present invention.

They are also solved by a graphical representation of a macromolecule and/or species, as defined above, generated by the method according to the present invention.

As used herein, the term “mechanical analogue” is meant to designate a device or apparatus or machine that is designed and constructed by man and performs a specific mechanical action. Examples for such a mechanical analogue are, without wishing to be restricted thereto, a cutter, a drill, a gear-box, a motor, a wing, an egg-whisker etc.

Such a mechanical analogue performs a specific function, and in order to perform such a function it needs to have specific parts, each part performing a specific component of said function. For example, a drill must have at least three parts, one which is actually responsible for creating a hole, i.e. the borer, one which generates the revolving motion of the borer, i.e. a revolving part, and c) a handle which allows the operator to hold the drill. It can sometimes be that b) and c) coincide.

As used herein the function of the mechanical analogue is meant to be “comparable to the function of the protein molecule”, if it provides for the same effect. For example, the cutting effect of a cutting machine is comparable to the cutting effect of a protease.

The methodology according to the present invention leads to a new set of experiments in protein research, e.g. aspartic proteinase research, which in turn may have direct impact on the concepts of structure based drug designing. The methodology systematically categorizes the structural complexity of macromolecules, e.g. proteins into simple functional components enabling better understanding. It further enables the researcher to apply the principles of tensegrity and mechanics to single macromolecules, e.g. protein molecules. This also introduces a new representation of proteins which is likely to provide a better understanding of protein structure and function and their relation in a way better than the existing representations. The representation of macromolecule architectural components (MACs) developed to graphically project the methodology is also efficient in projecting functional aspects of the macromolecule; furthermore the methodology according to the present invention shows new nanomolecular machines and has implications for novel nanomolecular manufacturing techniques.

Reference is now made to the figures, wherein

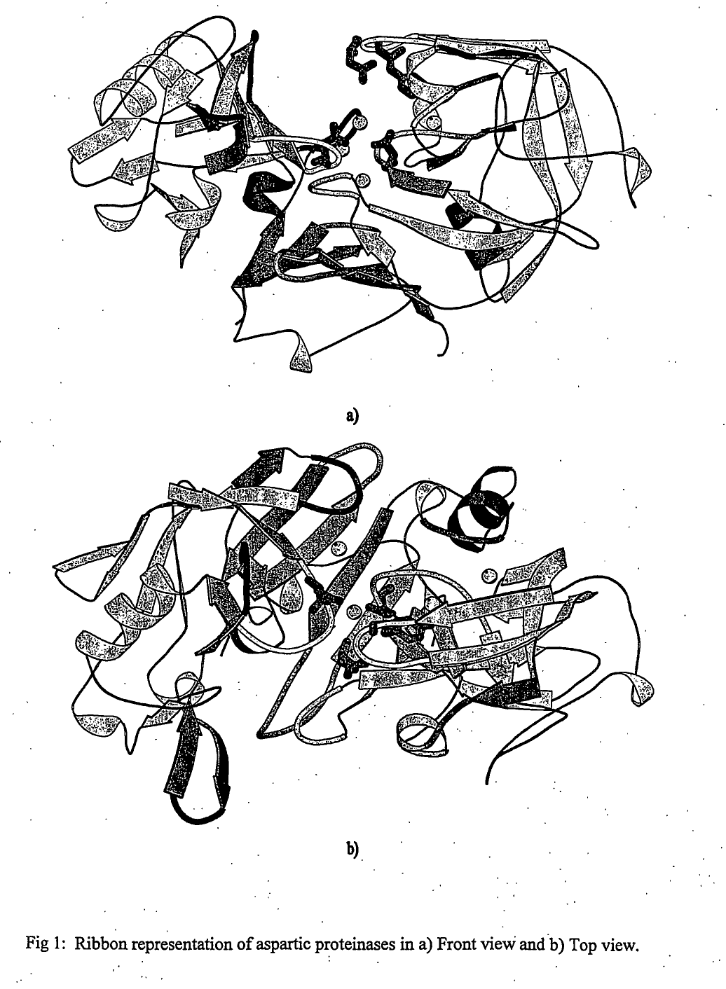

FIG. 1 shows a ribbon representation of aspartic proteinases in a) front view and b) top view,

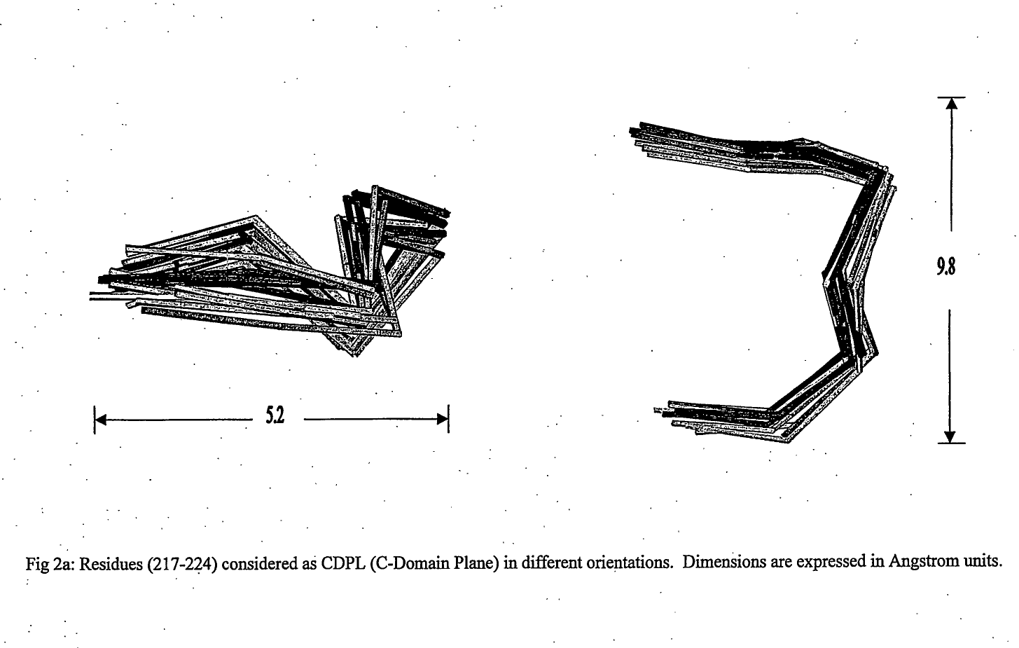

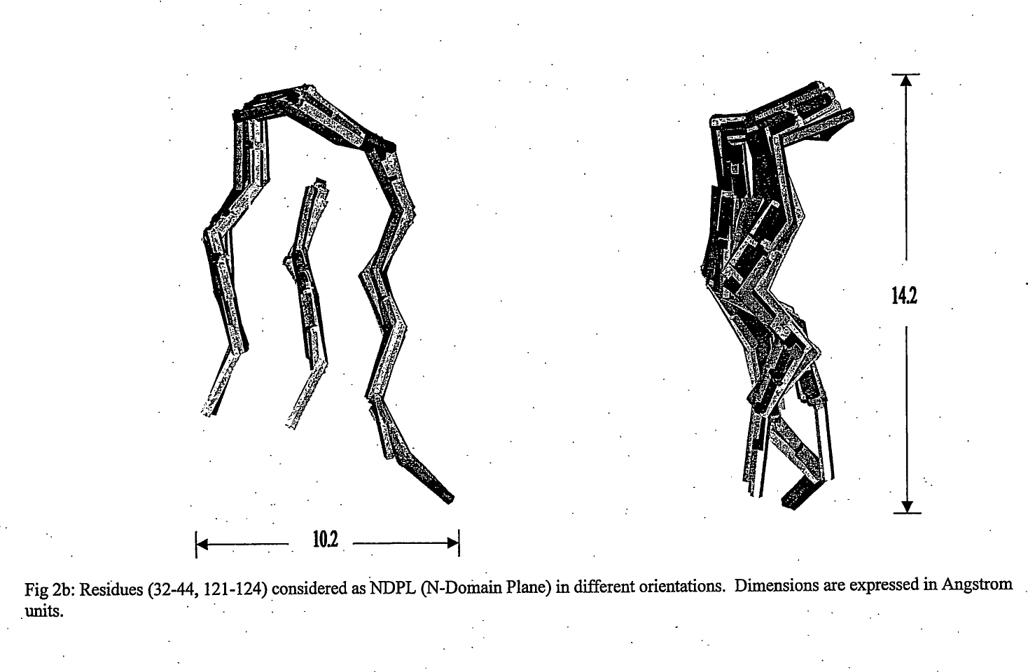

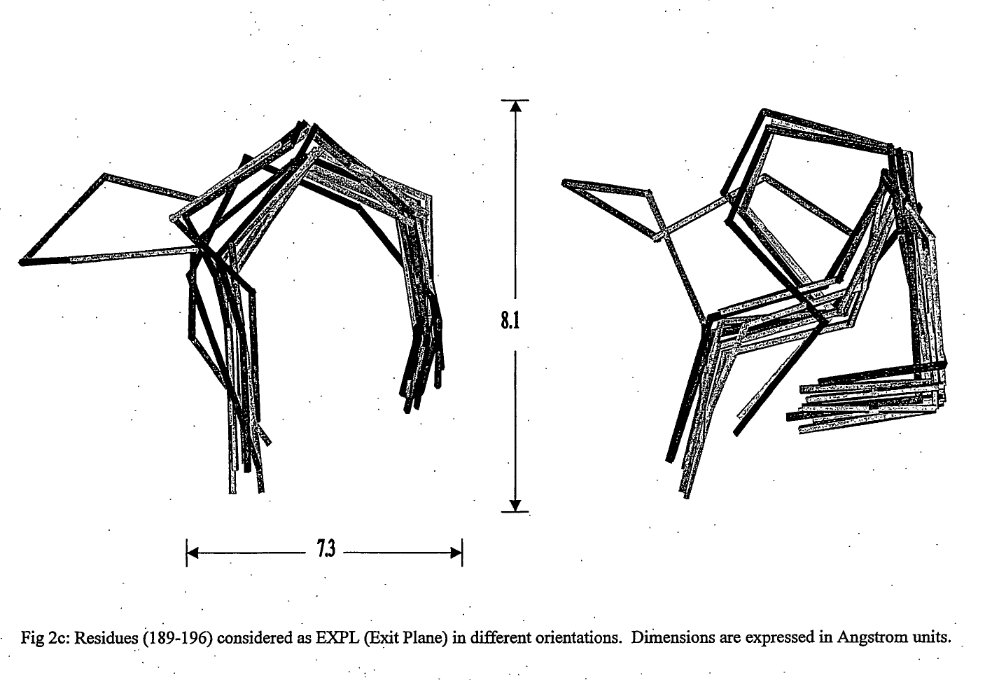

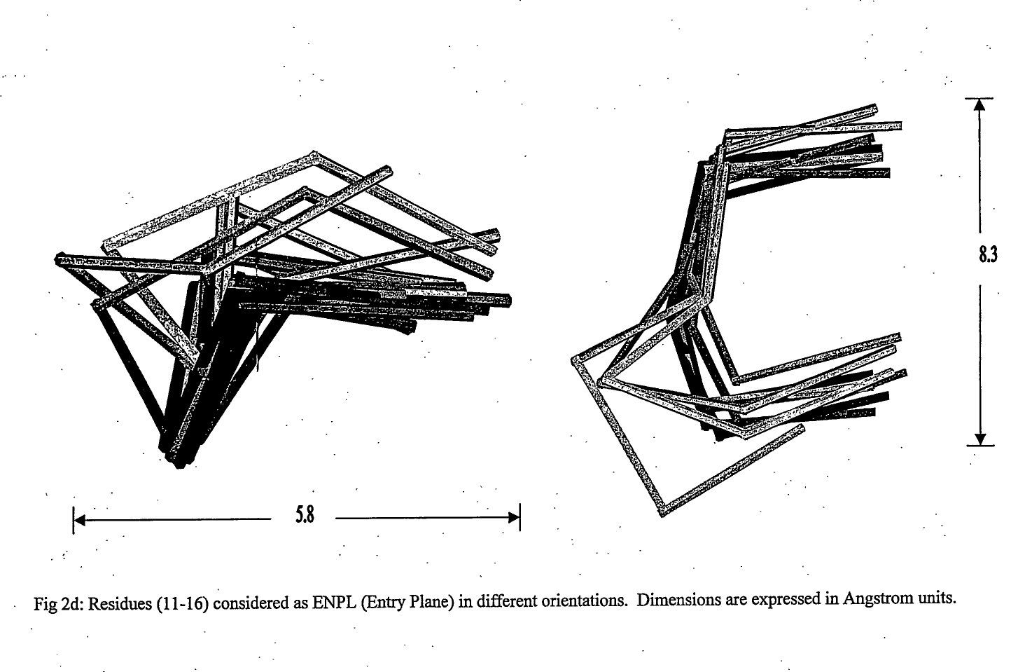

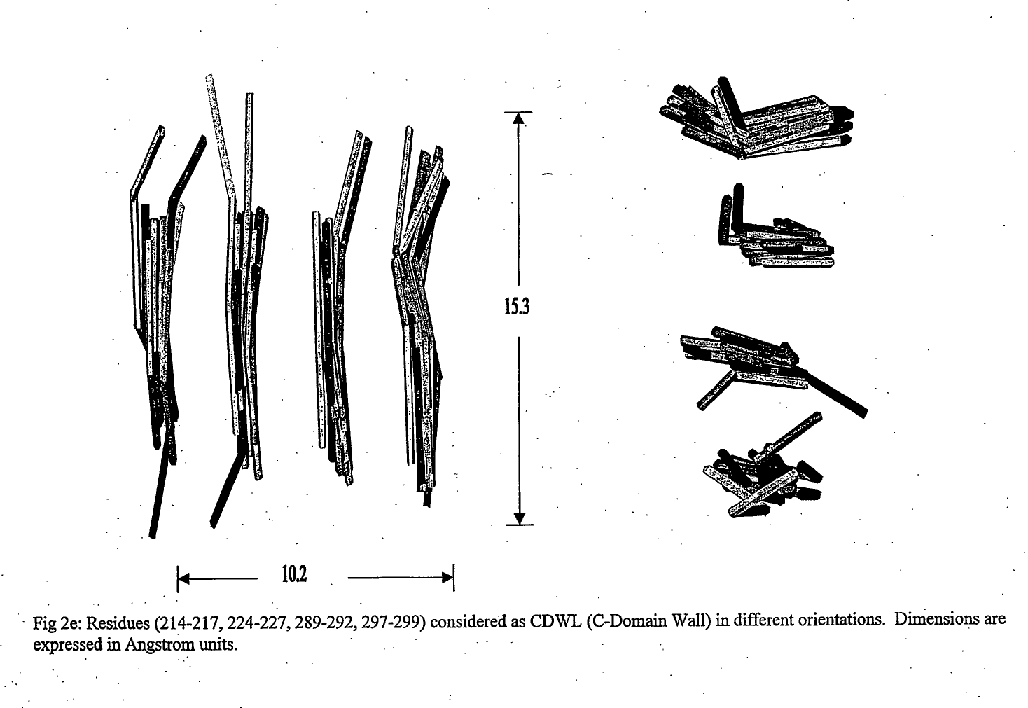

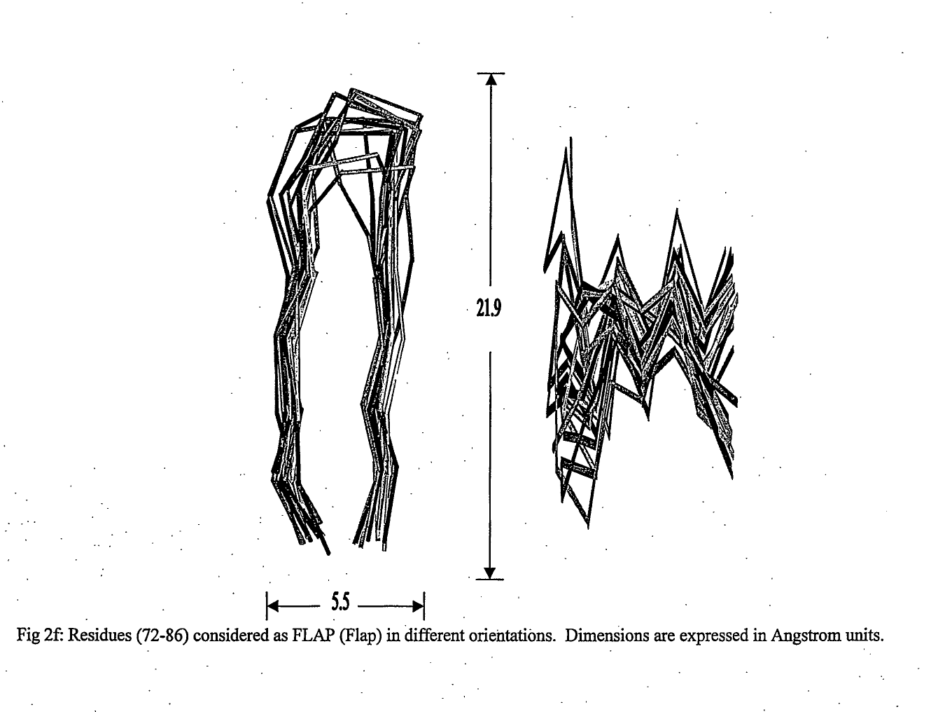

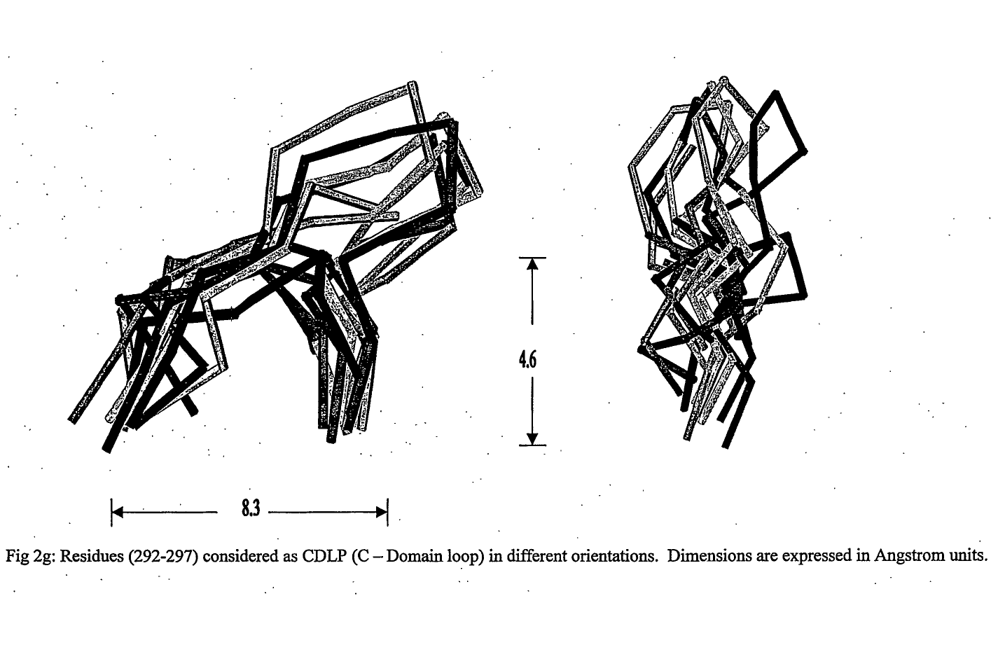

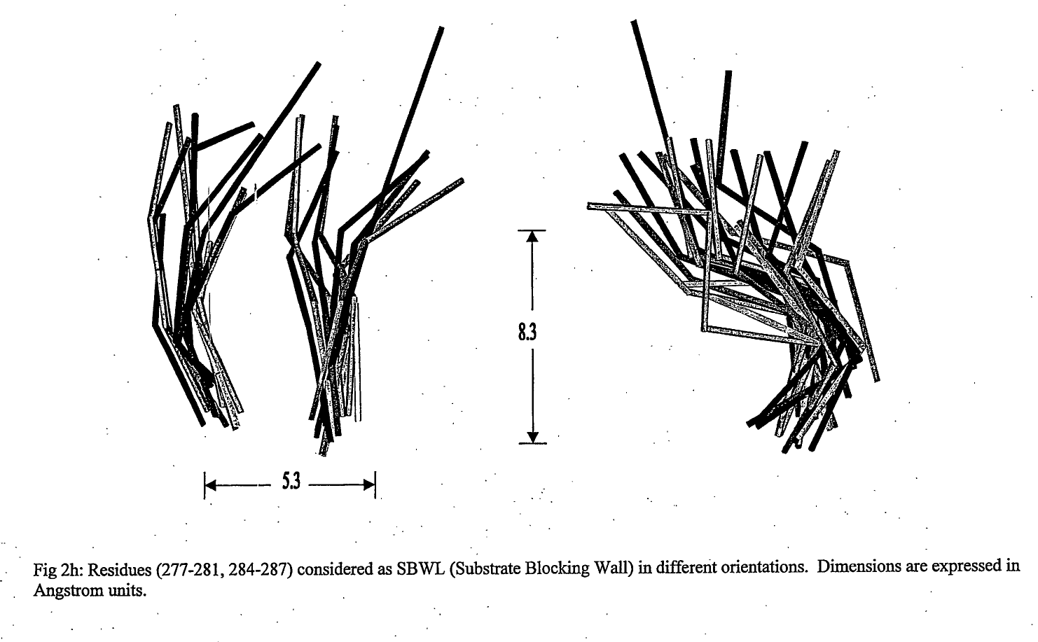

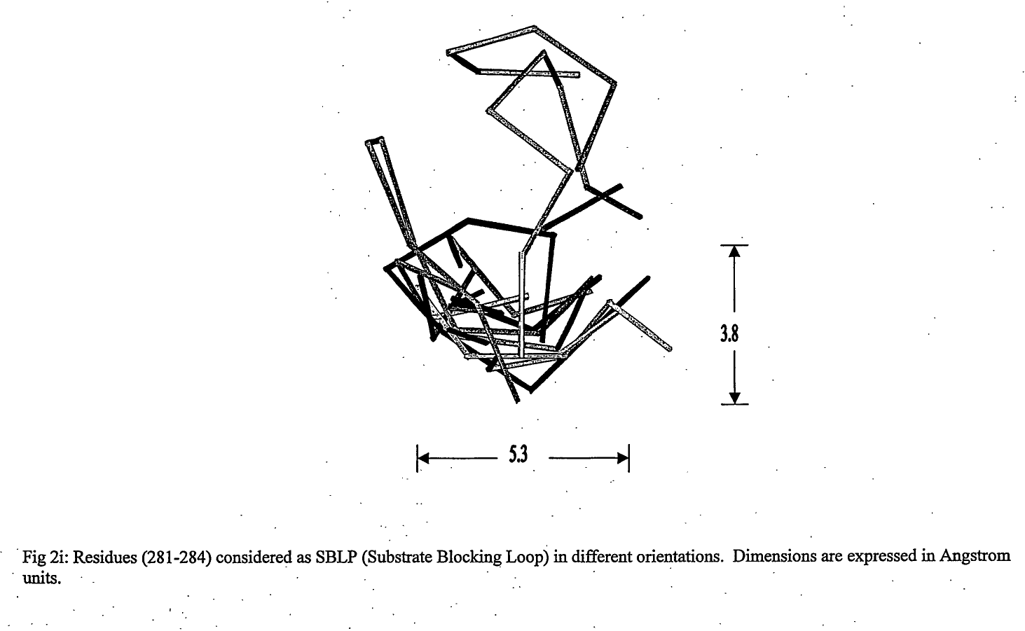

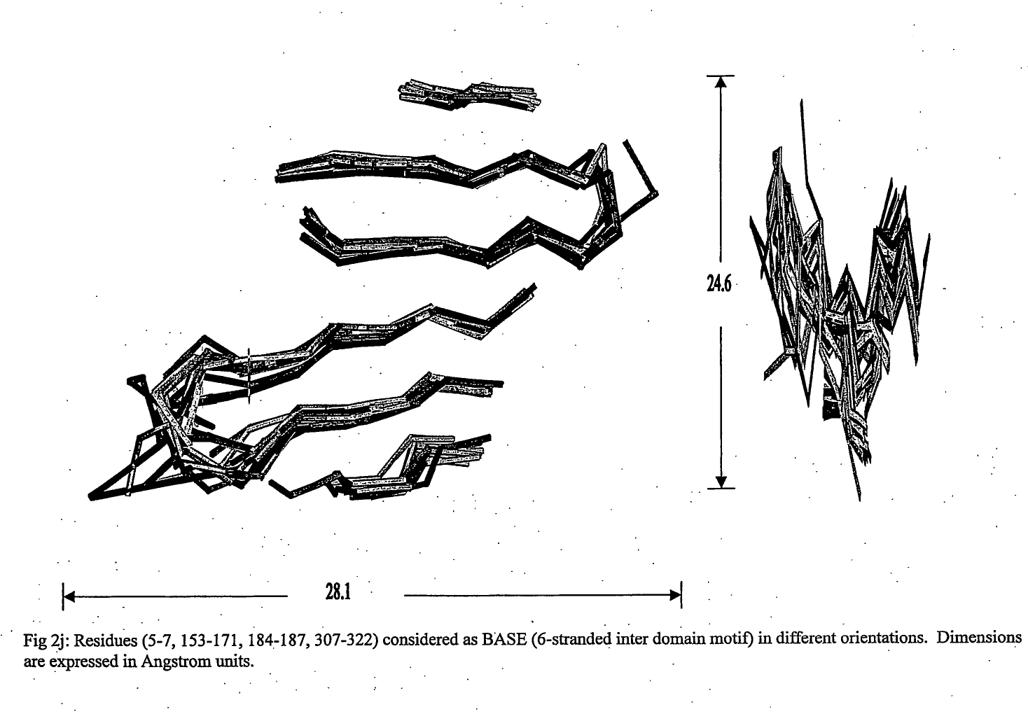

FIG. 2 shows the various protein architectural components of aspartic proteinases, in particular a) the C-domain plane (CDPL) in different orientations, b) the N-domain plane (NDPL) in different orientations, c) the exit plane (EXPL) in different orientations, d) the entry plane (ENPL) in different orientations, e) the C-domain wall (CDWL) in different orientations, f) the flap (FLAP) in different orientations, g) the C-domain loop (CDLP) in different orientations, h) the substrate blocking wall (SBWL) in different orientations, i) the substrate blocking loop (SBLP) in only one orientation, j) the base (BASE) in different orientations, with all dimensions in FIG. 2 being expressing in angstrom units Å,

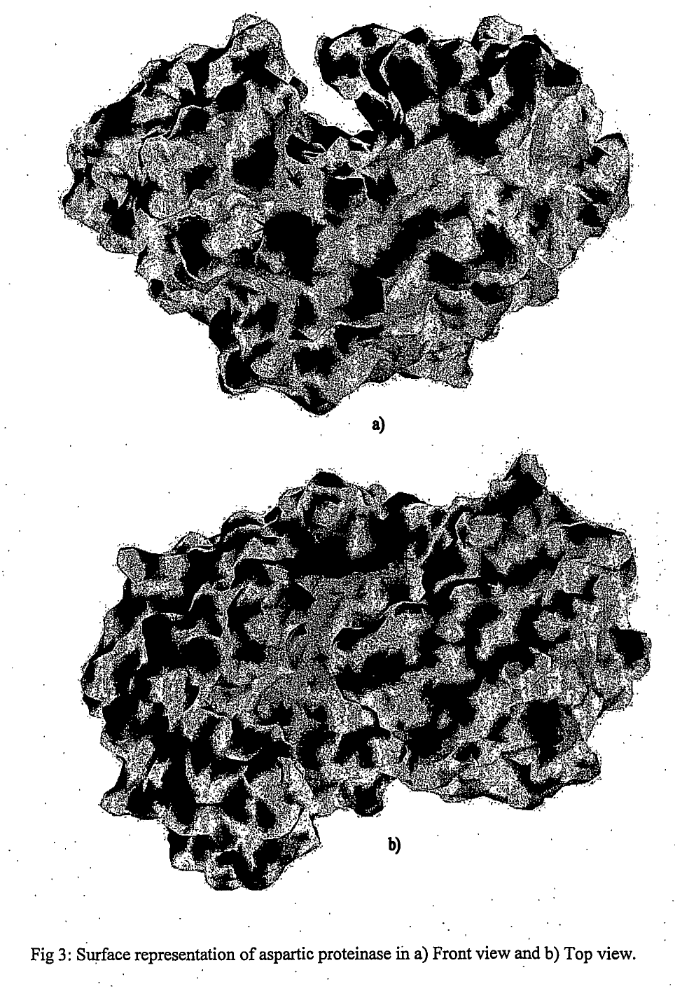

FIG. 3 shows a surface representation of aspartic proteinase in a) front view and b) top view,

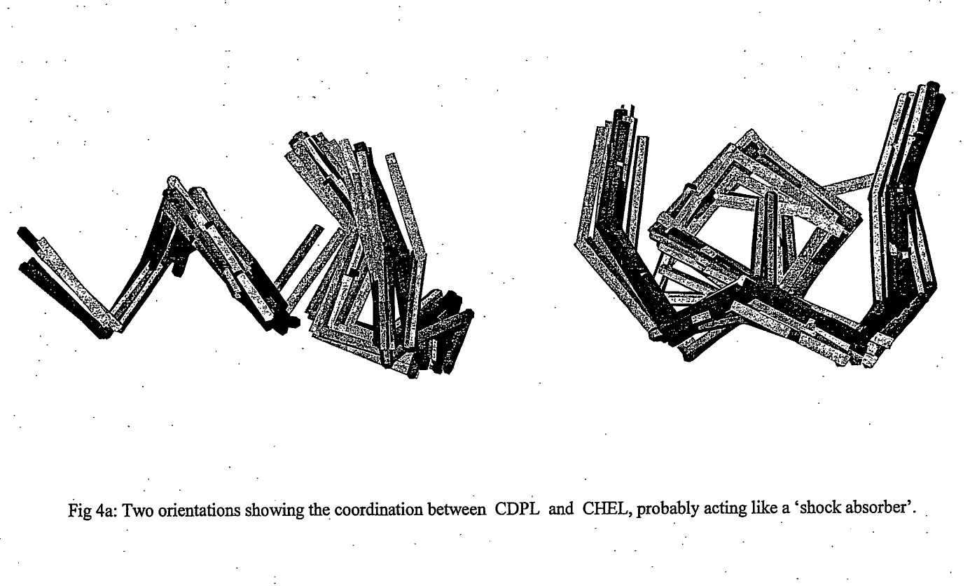

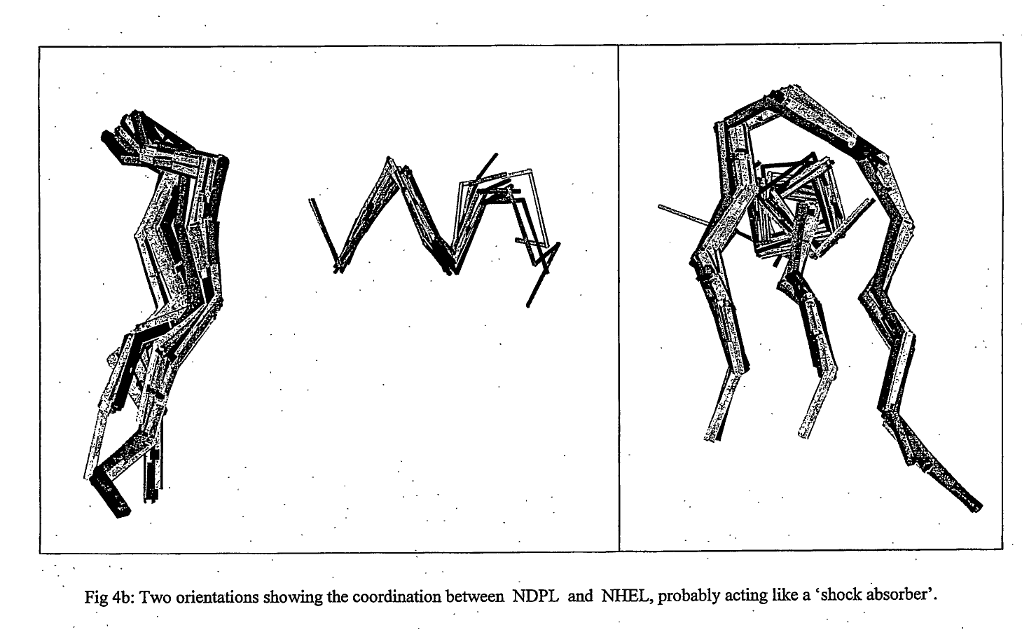

FIG. 4a) shows two orientations of the coordination between the C-domain plane (CDPL) and the C-terminal helix (CHEL), which act like a “shock absorber”,

FIG. 4b) shows two orientations of the coordination between the N-domain plane (NDPL) (thick line) and the N-terminal helix (NHEL) (thin line), which act like a “shock absorber”,

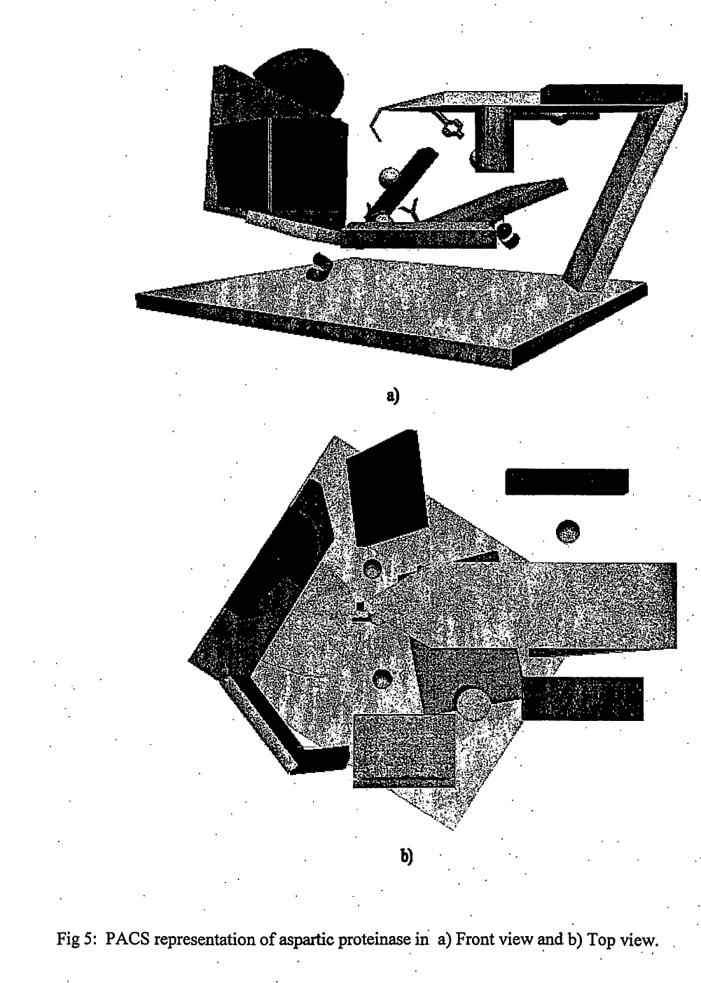

FIG. 5 shows a representation of all previously discussed macromolecule architectural components (MACs), in this case protein architectural components (PACs) of aspartic proteinase in a) front view and b) top view, and

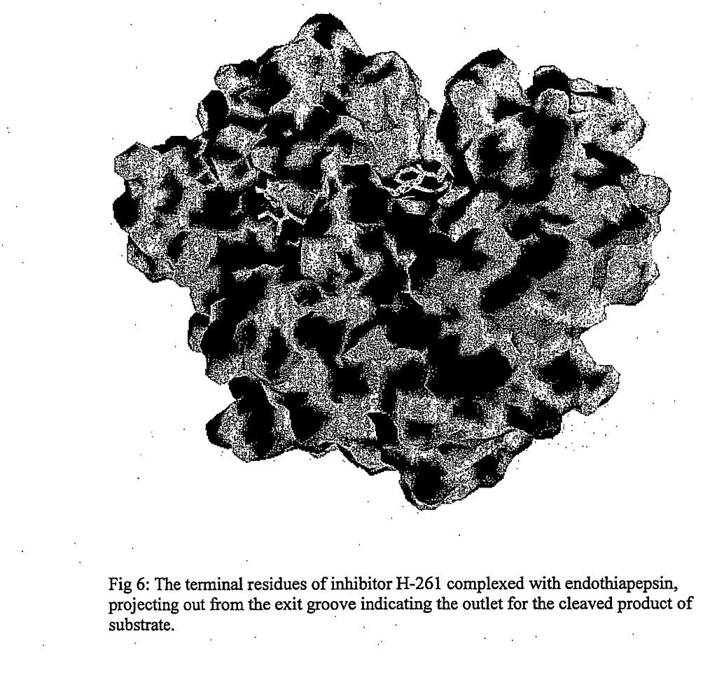

FIG. 6 shows the terminal residues of inhibitor H-261 complexed with endothiapepsin, an aspartic proteinase, which inhibitor is protecting out from the exit group indicating the outlet for the cleaved product of substrate.

The invention will now be further described by reference to the following examples which are given to illustrate, not to limit the invention.

### EXAMPLE 1

Methodology

Twelve native (uncomplexed) forms of aspartic proteinases were used in the present analysis for identifying MACs, in this case, since the macromolecule is a protein, for identifying Protein Architectural Components (PACs) (Table 1). All the native proteinases were superimposed using STAMP (Russell, R. B. & Barton, G. J. Struc. Funct. Gen. 14, 309-323 (1992)). The secondary structural elements, α-helices and β-strands, of the aligned proteinases were identified using hydrogen bonding criteria and were found helpful in identifying PACs more reliably.

Reverse Engineering Principles:

A fair amount of information can be retrieved by inspecting a machine or a mechanical component. Through knowledge of function, efficiency of work and critical inspection of the machine, it is possible to guess the different components involved, the underlying principle behind their usage and the information about the material used, with considerable accuracy and reliability. This procedure is called reverse engineering (Katheryn A. Ingle. (McGraw-Hill Professional Publishing, London; 1994)). A fundamental rule of thumb in machine design is that “every component should be flexible enough without permanent deformation and rigid enough to hold itself and its coordinating parts.”

A similar situation is likely to exist in the case of proteins. The finished machine in the form of a three-dimensional structure is available but, the design principles or concepts arc not known which presumably are used by the protein to achieve its architecture and specified function. However, biochemical, mutational and kinetic information available on proteins can be used to identify the PACs, which may play a key role in the pre-defined function of the protein.

General Principles of Structure:

A universal set of building rules seems to guide the design of biological structures—from simple carbon compounds to complex cells and tissues. This is reflected in the common set of elements, amino acids and nucleic acids used in building vast array of molecules that function in the living body. Symmetry is another universal principle which is utilized to minimize randomness in complexity. Almost all proteins have well defined secondary structural elements. Their positioning is specific. The concept of hydrogen bonds can be considered as the primary design principle that enables flexibility in the protein structure.

Identifying a Mechanical Analogy from the Protein's Function and Properties:

Aspartic proteinases take part in activities as diverse as gastric digestion (pepsin and gastricsin), maintenance of blood pressure (renin), milk clotting (chymosin), protein turnover (cathepsin D), parasite viability (retropepsins and plasmepsins) etc. Their involvement in the life cycle of disease-causing organisms has made them potential targets for developing therapeutic agents against fatal diseases such as AIDS¹¹. They are involved in the hydrolysis of the substrates through general acid-base catalytic mechanism. Thus, aspartic proteinases can be compared to macroscopic cutting machines.

Identifying the Essential Parts of the Mechanical Analog:

The following can be considered as the essential parts of a simple cutting machine.
i) Cutting Blade: this can be considered as a sharp edged material, in chemical terms it is expected to be a very reactive group.
ii) Cutting Space: In general, the space should have boundaries in all the directions to provide support for the material to be cut. It can be considered as a hollow cube. It is expected that the ‘cutting blade’ projects into this ‘cutting space’.
iii) Entry and Exit ports for the material and its cleaved components: To enter into and exit from the cutting space, there should be an ‘entry gate’ and ‘exit gate’. These gates should be comparable to the size of the material which has to be cut. Hence the entry and exit gates should be extensions or parts of the ‘cutting space’.
   Identifying the PACs from the Mechanical Parts:

The PACs in aspartic proteinases were identified, which play the role equivalent to the above mechanical components, by visual inspection of all the aligned proteinases using O (Jones, T. A., Zou, J. Y., Cowan, S. W. & Kjeldgaard, M. Acta Cryst. A47, 110-119 (1991)), Swiss-Pdb Viewer (Guex, N. & Peitsch, M. C. Electrophoresis 18, 2714- 2723 (1997)) and Molscript (Kraulis, P. J. J. Appl. Cryst. 24,946-950 (1991)) programs. The angles between the different PACs were calculated using the direction cosines (DCs) of each PAC. DCs of the helical axes of all the aligned proteinases were calculated using functions explain the functions here following the methodology described elsewhere (R. Srinivasan, R. Balasubramanian & S. S. Rajan. J. Mol. Biol. 98, 739-747 (1975)). The DCs for the best fitting planes for a given set of Cα atoms were calculated using the Singular Value Decomposition (SVD) technique (Craig M. Shakaii. J. Res. Natl. Inst. Stand. Technol. 103; 633-637 (1998)). SVD is a powerful technique which can deal with solving equations that are either singular or dose to singular. SVD is the method of choice for solving linear least-squares problems, since it is suitable for eliminating data points that are too much offset from the remaining data.

A difference vector matrix is formulated for each PAC of each native proteinase such that,
‘A’=[(xᵢ−<x>) (yᵢ−<y>) (zᵢ−<z>)]>)]
(xᵢ, yᵢ, zᵢ)=[X, Y, Z] coordinates of a Cα atom in the PAC
(<x>, <y>, <z>)=(Σxᵢ/n, Σyᵢ/n, Σzᵢ/n)
n=Number of Cα atoms in each PAC.

The matrix ‘A’ was solved by the singular value decomposition method (SVD) using the function, svd(), provided in the program Octave (Octave (1998) httv://www.octave.org/). The singular vector corresponding to the smallest singular value of the diagonal matrix represents the direct cosines DCs [l,m,n], of the best-fitting plane for the coordinates under consideration and hence the [l,m,n] of the PAC. The DCs for PACs of all the proteinases were noted. The angle between the PACs were then calculated using the formula,
θ=cos⁻¹ (l₁l₂+m₁m₂+n₁n₂).
for the case of two PACs with [l₁, m₁, n₁] and [l₂, m₂, n₂] being the direction vectors of a PAC₁ and PAC₂ respectively. The angles thus calculated and listed in tables are the angles between the normals to planar components and/or the helix axes. The average angle and standard deviation (SD) of each PAC for all the native proteinases were also calculated. The atomic displacement parameter (B-factors) of Cα atoms of all the native proteinases were normalized (Parthasarathy, S. & Murthy, M. R. N. Protein Science. 6, 2561-2567 (1997)). The average B-factor of each PAC in proteinase was noted. The sequence identity between the entire sequences of proteinases was obtained using MALIGN (Martinez, H. M. Nucleic Acids Res. 16, 1683-91 (1988)). Similarly, the equivalent PAC sequences were aligned and the sequence identity was noted. All equivalent PACs were structurally aligned using the program STAMP.

It should be noted that as the number of coordinates decreases, the reliability on the STAMP score also decreases. Similarly, the greater the number of coordinate points, the more reliable are the DCs obtained for the best fitting plane. As the components being identified have less number of points, care has been taken to visualize each component on graphics as well and interpret the results. FIG. 2 and Table 2 are used to complement each other in judging and identifying the PACs and further interpretations.

General Graphical Representation:

The ribbon representation of proteins (FIG. 1a (Front view of aspartic proteinase) and FIG. 1b (Top view of the aspartic proteinase) has been taken as the reference for the following discussion in this paper. The PACs are represented either as ‘planks’ or ‘springs’ or ‘cylinders’, whose dimensions are approximated from the identified components. FIG. 2 shows the different PACs identified and their dimensions (Length and Breadth, the thickness is considered as constant). The [l,m,n] and the centroids (centre of mass) of the individual PACs were used to draw a schematic diagram (FIG. 5a & b) using Blender (Blender: 2.23 Version, http://www.blender3d.com) version 2.23 program.

According to the structural classification of proteins (SCOP) (Murzin, A. G., Brenner, S. E., Hubbard, T. & Chothia, C. J. Mol. Biol. 247, 536-540 (1995)), aspartic proteinases belong to all β protein class, acid proteinase fold and acid proteinase super family. The super family is further categorized into pepsin-like and the retroviral families. In total, acid proteinase super family has approximately 280 crystal structures, of which nearly 70% are the crystal structures of HIV-inhibitor complexes. At present, crystal structures of aspartic proteinases from 21 different sources are available in pepsin-like family. Similarly, crystal structures of retropepsins from 7 sources are available. However, only 15 native structures (12 structures from pepsin-like family, 3 structures from retroviral family) are known. The others being either complexes or mutants or zymogens. The native structures are used in the present analysis.

The members of the pepsin-like family are monomeric and the overall secondary structure consists almost entirely of pleated sheet with very little a-helix. Typically, the proteinases have about 325 amino acid residues which form two lobes and a pseudo dyad axis of symmetry relates the two lobes. There is a high structural homology between the members as against sequence identity (for a review, see (David R. Davies. Annu. Rev. Biophys. Biophys. Chem. 19,189-215 (1990)) (Table 1).

### EXAMPLE 2

Protein Architectural Components (PACs) in Aspartic Proteinases:

a) Equivalent of ‘Cutting Blade’:

Similar to the properties of the ‘cutting blade’, two aspartates (Asp35, Asp218, rhizopuspepsin numbering) one from each lobe, are involved in the catalysis of the substrates. When a cutting blade is not being used, it is generally covered to avoid unnecessary cutting of other material and to avoid decay of the blade sharpness due to the environmental conditions. Similarly, the highly reactive carboxyl groups of the two aspartates are stabilized by a conserved water, Wat507 (rhizopuspepsin numbering), when there is no substrate. The position of Wat507 is replaced by carbonyl oxygen of the substrate during substrate binding.

b) Equivalent of ‘Cutting Space’:

The substrates or inhibitors bind in the space available between the N- and C- terminal lobes. This is called the active site cleft, equivalent of ‘cutting space’ and is about 40 Å long, running across the molecule separating the two lobes (David R. Davies. Annu. Rev. Biophys. Biophys. Chem. 19,189-215 (1990)). A more precise description is attempted by characterizing the PACs which form the active site cleft. It is further analyzed to quantify the length, breadth and height of the active site cleft.

Work Bench of the Active Site Cleft Formed by ‘NDPL’ and ‘CDPL’:

The two active aspartates responsible for catalysis project into the active site cleft from two loops, residues 217-224 and residues 32-44, 121-124. These loops are related to each other by the pseudo dyad axis and are held together by hydrogen bonds, described as ‘fireman's grip. These 2 loops look like two ‘planks’ separated by a distance of approximately 5.5 Å. The 2 aspartates project from the edges of the ‘planks’. These ‘planks’ are considered as PACs and named as ‘NDPL’ and ‘CDPL’. By analogy, their function is to form the ‘work bench’ for the catalysis through supporting the ‘chemical blade’. The angle between the normals to these 2 PACs is 54.6° (SD 3.4°) (Table 3). The relatively low SD indicates the consistently maintained and hence conserved angle between the two PACs. Their average normalized B-factors are −0.661 and −0.823, respectively (Table 4). The B-factors of these two components are among the lowest, indicating the stability of these two components across the acid proteinase super family. CDPL and NDPL, together form the bottom of the active site cleft. The components are structurally well conserved (FIG. 2(a & b) and Table 2(a & b)).

Rear and Front Faces of the Cleft Formed by ‘EXPL’ and ‘ENPL’:

The substrate has to be stopped from slipping away from the active site after entering into the active site cleft and passing over the ‘work bench’. An verticed loop (residues 189-196) is seen at about 12 Å (distance between the centroid of the loop and the active site center which is taken as Wat507) from the active site into the rear side of the proteinase (FIG. 1). The role of blocking the substrate from slipping is attributed to this loop and is considered as another PAC. It is named as ‘EXPL’. ‘EXPL’ has deletions (porcine pepsin, 4PEP) and insertions (for example, pusillopepsin, 1 MPP) when compared to fungal proteinases (FIG. 2c, Table 2c), hence, inconsistency is observed in the angles between ‘EXPL’ and other components. From Table 3, the angle made by this component with another component, ‘BASE’ (discussed later), clearly forms two groups. The first group constitutes the fungal proteinases, viz, 2APR, 3APP, 4APE, 1 MPP show an average angle of 50° between the normals of ‘EXPL’ and ‘BASE’. The exception being 2ASI which shows an angle of 25° with ‘BASE’. The other group, mammalian proteinase group has 4PEP, 1AM5, 4CMS, 1B5F, 1BBS, 1LYA, 1PSN, show an average angle of 25°. Similar grouping is seen when the angles are compared with other PACs also. Hence, the SD in the angle between ‘EXPL’ and other components is relatively high. This clear demarcation into fungal proteinase group and mammalian proteinase group might be a clue to explore this region to understand the substrate specificities between mammalian and fungal proteinases. The average normalized β-factors are relatively low indicating that this component is considerably stable. It is observed that ‘EXPL’, ‘NDPL’ and ‘CDPL’ are held together through strong hydrogen bonds by Wat502, which is a totally buried invariant water and is approximately 8.5 away from the active site centre and into the rear side of the proteinase (Prasad, B. V. L. S. & Suguna, K. Acta Cryst. D58, 250-9 (2002)). This places ‘EXPL’ in between ‘NDPL’ and ‘CDPL’, away from the active site centre and into the rear side of the proteinase. The 0.0 accessibility of Wat502 indicates the closure of the cleft from the rear side by ‘EXPL’.

Similar to ‘EXPL’, its pseudo symmetry related β-hairpin loop (residues 11-16) seems to be the entry port for the substrate. The interesting difference of this loop from ‘EXPL’ is that it is not erect. It forms a plane which is almost parallel to the ‘BASE’. If this loop were similar to ‘EXPL’, the substrate will be blocked from entry into the active site. Hence, this looks like forming the bottom of entry gate for the substrate, prior to moving onto the work bench. This is therefore considered as another PAC and is called ‘ENPL’. This component is approximately 12.5 Å in front of the active site centre. The angle made by this component with others can be clearly separated into three groups (Table 3), similar to that noted for ‘EXPL’. First group constituting the proteinases, 2APR, 3APP, 4APE, show an average angle of about 15° with ‘BASE’. Mucorpepsin (2ASI) and pusillopepsin (1MPP) are forming another group with an average angle of 69° due to the insertions clearly seen in FIG. 2d & Table 2d. The third group constitutes the mammalian aspartic proteinases, which have 23° on average. Hence, the SD is high for this component. As seen from the figure, planarity is maintained in all except 1MPP and 2ASI because of insertions in this region. When compared to ‘EXPL’, the average angle ‘ENPL’ makes is around 15°, supporting the possibility of this component to be a platform for the substrate before entering the active site cleft. Moreover, all the other sides of the cleft are blocked by other PACs. Similar to the role played by Wat502 in stabilizing the rear region of the active site cleft, Wat513, the pseudo symmetry equivalent of Wat502 also forms strong hydrogen bonds with. ‘CDPL’, ‘NDPL’ and ‘ENPL’, thereby stabilizing the front region of the active site cleft (Prasad, B. V. L. S. & Suguna, K. Acta Cryst. D58, 250-9 (2002)).

Left Face of the Eleft:

On careful examination, it is identified that on the left side, 4 strands (214-217, 224-227, 289-292, 297-299) line up forming a planar structure as shown in FIG. 1b & 2e. The role attributed to this plane is to provide the left side boundary wall for the active site cleft. Besides the above role, it could also direct the substrate towards the exit as suggested from the surface representation (FIG. 3b). With these roles to play, this group of strands together are considered as a PAC and named as ‘CDWL’. The very low B-factors and low SD indicate that ‘CDWL’ is a highly conserved and stable component (Table 4). The angle between ‘CDWL’ and ‘CDPL’, 52.1°, is highly conserved as seen from the relatively low SD (34°) (Table 3).

Right Face of the Cleft:

The right side of the active site cleft has been well characterized in terms of its role in substrate binding. The active site cleft seems to have extended into the N-terminal lobe. This lobe is stabilized by a number of structurally and functionally important hydrogen bonds between the active aspartates, ‘NDPL’ residues, ‘FLAP’ residues (discussed in the following subsection) and invariant waters. The perpendicular distance between the ‘FLAP’ and ‘NDPL’ is around. This region is filled with invariant waters such as Wat5IO, Wat505 and Wat508 (Prasad, B. V. L. S. & Suguna, K. Acta Cryst. D58, 250-9 (2002)). These waters form a network of hydrogen bonds connecting the active aspartates, Wat507, ‘NDPL’ residues, ‘FLAP’ residues and a few residues surrounding the region. The two strand segments, residues 65-72 and 86-91 are hydrogen bonded and form a long sheet. Is it shown in the figures. This sheet is perpendicular to and supports one end of the ‘FLAP’. This sheet is on the extreme right of the proteinase closing the right face of the extended active site cleft. Interestingly, additional strength is extended to the base of the sheet by a conserved hydrogen bond bridge between Thr 65 O, Asn 91 OD1 and two invariant waters, Wat592 and Wat617 (Prasad, B. V. L. S. & Suguna, K. Acta Ciyst. D58, 250-9 (2002)).

Top of the Cleft:

The ‘FLAP’ is another PAC (typically, residues 72-86), which is in the N-terminal lobe and closes over the substrate when it binds. Flap is flexible in native structures and appears more tight and rigid in the complexes. These observations are supported by its comparatively high B-factors in ‘open’ and ‘closed’ (complexed) conformations. The recent insights obtained through study on the role of invariant waters in the pepsin4ike family also support that the flap acts like a biological ‘cantilever’. It is observed that in the complex, the hydrogen bond between Wat510 (rhizopuspepsin numbering) and Tyr 77 OH becomes stronger compared to native structures (Prasad, B. V. L. S. & Suguna, K. Acta Cryst. D58, 250-9 (2002)). The ‘FLAP’ is placed almost perpendicular and above the active site center at a distance of about 12 Å. The ‘FLAP’ has been the subject of many mutational studies for understanding its role and has been reported as very essential for the catalysis of the substrate. The B-factors of the ‘FLAP’ are relatively high (Table 4). The structural alignment clearly shows that the tip of the ‘FLAP’ is highly flexible (FIG. 2f & Table 2f). Similar to ‘CDPL’ and ‘NDPL’ forming the bottom of the active site cleft, ‘FLAP’ and another highly flexible and variable loop (292-297) together play role in closing the cleft from the top. The variable loop is highly flexible and has insertions and deletions (FIG. 2g & Table 2g). The high B-factors and the high SD support the flexible nature of this loop. This loop is implicated in the specificity of the substrate. It is considered as another PAC and is called ‘CDLP’. The angle between ‘CDWL’ and ‘CDLP’ is 83.229° with high SD (Table 3). This is not only due to the insertions and deletions in this component but also due to the inherent high flexibility of the residues. It is also reported that this polypeptide segment is generally disordered (Cele Abad-Zapatero, Timothy J. Rydel, & John Erickson. Proteins: Struc. Funct. Gen. 8, 62-81 (1990)).

Entry of the Substrate:

Two components are identified which might function in regulating the entry of the substrate, in stabilizing the substrate binding, optimal positioning and blocking the substrate from reverse movement. They are named as ‘SBWL’ and ‘SBLP’. ‘SBWL’ and ‘SBLP’ are continuous. ‘SBWL’ is considered as a ‘small plank’ formed with two.small strands (277-281, 284-287). They are strands in some proteinases (2APR) and random coils in some proteinases (4PEP) (FIG. 2h & Table 2h). ‘SBLP’ is the loop joining the two strands of ‘SBWL’. Similar to the insertions and deletions observed in ‘EXPL’, ‘ENPL’ and ‘CDLP’, it is noticed that ‘SBLP’ (281-284) has insertions (Table 2i & FIG. 2i). The residues involved in these two components are reported to be among the highly disordered residues in aspartic proteinases (Cele Abad-Zapatero, Timothy J. Rydel, & John Erickson. Proteins: Struc. Funct. Gen. 8, 62-81 (1990), Andrej Sah, Veerapandian, B., Jon B. Cooper, David S. Moss, Theo Hofinann & Tom L. Blundell. Proteins: Struc. Funct. Gen. 12, 158-170 (1992)). This loop contains mostly ionizable residues (Table 2i). From the high B-factors and high deviations, it may be expected that these two components are always in movement. They both maintain an average angle of 56.7° between them. The ‘SBLP’ is on the left side of ‘ENPL’ and a residue segment (110-118) is on the right side. The distance between SBLP and this segment probably decides the breadth for the entrance at any instance of time. The average breadth is 14 Å which is comparable and little larger than the typical breadth of a substrate (12.5 Å) and allows the substrate to enter. This is clearly seen from the surface representation (FIG. 3a & b).

The other possibility for the substrate to enter the active site cleft is through the gap between ‘CDLP’ and ‘FLAP’. The typical distance between these two components is around which suggests that unless the ‘FLAP’ or ‘CDLP’ or both open outwards, a substrate or inhibitor cannot enter.

Safety and Flexibility Considerations:

A Safety for the Work Bench Through Another Protective Shield by ‘BASE’:

One of the most conspicuous component in aspartic proteinases is the ‘BASE’(5-7,153-171,184-187,307-322), a ‘6 stranded inter-domain β-sheet’ in pepsin-like family and 4 stranded β-sheet in retroviral family of proteinases (David R. Davies. Annu. Rev. Biophys. Biophys. Chem. 19,189-215 (1990)). FIG. 2j & Table 2j show that this component is highly conserved. Each lobe contributes three strands in pepsin-like family. This has been called variously as ‘base’, ‘floor’, β-pleated sheet’, ‘central motif’ etc. This component lies beneath the work bench. This may be considered as the ‘protective shield’ for the aspartic proteinase. The role of ‘BASE’ could be to-keep-the-work-bench ‘safe’. This is the largest component, almost spanning an area of 25×25 Å².

Helices as Shock Absorbers:

An interesting observation has been that there are 2 helices, ‘CHEL’ and ‘NHEL’, which are almnost perpendicularly positioned under the CDPL and NDPL respectively (FIG. and Table 2(k & 1)). This could be a strategy to avoid collapse of CDPL or NDPL under excessive stress from the substrate. NHEL and CHEL are thought to act like ‘shock-absorbers’. These helices are not only seen in pepsin-like family but also in retroviral proteinases which further strengthen the role assigned to these helices as ‘shock absorbers’. The angle between both helices is 54.2° (SD 6.8°), almost comparable to the angle between ‘NDPL’ and ‘CDPL’ (547°) The angle between the CHEL/CDPL is 18.4° (FIG. 4a). Due to the exceptionally high angle shown by endothiapepsin, the SD is high, otherwise the average angle is around 13°. Similarly, the average angle is around 12° for NHEL/NDPL if 4APE, 3APP and 1LYA are not considered (FIG. 4b). These exceptions could indicate the stress tolerant capacity of the proteinase to different substrates. This combination of a ‘plank’ and a ‘spring’ also suggests the probable existence of higher order PACS.

Presence of a Bushing Like Structure on Either Side of the Flap:

It is observed that on either side of the ‘FLAP’, two residue segments (108-111) and (130-133) are positioned in such a way that the ‘FLAP’ is maintained in place for efficient catalysis. This is similar to the use of a bushing in machines to keep the shaft in position and to avoid the slippage and wearing of the shaft. The segment 130-133 is more flexible than its equivalent on the other side of the ‘FLAP’ and is held by an invariant water, Wat746, found in all the pepsin-like aspartic proteinase family. The segment 108-111 is a strand and is directly hydrogen bonded to the FLAP.

Discussion:

Structural Classification has Limitations

The methodology according to the present invention has shown that the existing structural classification has its limitations in that it only positions the various secondary structural elements with respect to one another. In contrast thereto the method according to the present invention emphasizes the function and categorizes the protein structure on the basis of function. FIGS. 5a & b show the schematic representation of all the PACs identified placed at their respective centroids and relative angles. The functional aspects of the components are reflected with greater clarity and the function of the aspartic proteinase is seen with newer insights.

Exit of the Cleaved Products:

It should be noted that ‘EXPL’ thickness but not the surface of the plane is turned towards the active site centre (FIG. 1a & b); A free space of 13 Å wide (Arg 192 O Tyr 77 N) is seen between EXPL, FLAP and a 5 residue segment (127-131). A groove is seen in the surface representation (FIG. 3a & b). Additionally, there is no gap between ‘CDWL’ and ‘EXPL’. These observations suggest that substrate will be turned towards right and the cleaved products would probably exit through this groove. The crystal structure of endothiapepsin with H-261 inhibitor (PDB code: 2ER7) shows some portion of inhibitor projecting out through this groove (FIG. 6).

Optimal Positioning of the Substrate:

Using the PACs described above, the substrate entry, the optimal position for efficient catalysis and the exit of cleavage products can be understood, which complements the static picture of the 3D structures. At any particular instance of time, when ‘SBLP’ moves out, which is quite possible because of its highly flexible nature, the substrate enters the active site cleft. It is calculated that the angle between the ‘BASE’ and the workbench as a whole is 13.9° (SD 3.4°). The substrate need to travel in the substrate groove a distance of 25-30 Å up a gradual scope provided by the work bench. Being the left side boundary wall, ‘CDWL’ blocks and redirects the substrate towards the exit groove. This distance is approximately 20 Å which is approximately 3 residues. It can clearly be visualized by the surface representation of the aspartic proteinase (FIG. 3a & b) that the groove bends with an angle of 130-135°. Interestingly, it is observed that during the movement of substrate towards the exit, the substrate has to climb up another steeper scope which is due to the 31.5° (SD 2.9°) angle between the workbench and the ‘NDPL’. This is calculated to be approximately 5.22 (10×sin (31.5°)—where 10 is the approximate width of ‘NDPL’ and forms the hypotenuse of the trigonometric triangle) lift of the substrate residues. This relatively steep scope provides another strong support that the rear side groove probably may not be acting as an entrance. It is reported earlier that three residues of the substrate, represented as P1′ to P3′, can be accommodated in the rear side of the active site centre which is in accordance with the above observation.

Once the substrate turns and starts climbing the scope, the substrate will be pushed towards the N-terminal lobe resulting in positioning of substrate exactly above the CDPL aspartate.

This results in the downward movement of this edge of the component. Consequently, the opposite edge is pushed up. The ‘CDPL’, ‘CDWL’ and ‘CDLP’ are connected to each other in series and the included angles being 52.1° and 83.3° respectively. The upward movement of ‘CDWL’ moves the ‘CDPL’ towards flap. This description of coordinated movement of C-terminal lobe components agrees well with the earlier observations that the inter lobe angle in aspartic proteinases decreases on inhibitor binding. The coordinated movement of the three components pushes the substrate further into N-terminal lobe, now positioning it between the two active aspartates and presumably the catalysis is initiated at this instance. Besides this, the bending increases the strain on the substrate back bone making the substrate more liable to break down. This is probably a strategy to improve the efficiency of catalysis.

Due to the strain in substrate back bone and in an attempt to return to the original extended conformation, the terminal residues of the substrate, represented as P7 or P8, will be pushed into the region between SBWL and SBLP. The repositioning of the P7/P8 residues not only reduces the strain on the backbone to a small extent, but also probably places the carbonyl oxygen more optimally over the active aspartates. This displacement of terminal residues stops the substrate from reversing back due to the slope which the substrate climbs. This final displaced position can be seen in almost all the enzyme-inhibitor complex crystal structures.

If the substrate is longer, the terminal substrate residues will be hanging from SBWL and SBLP edge or will be pushed out through the ‘exit groove’(e.g., PDB 2ER7). This results in a restriction over the adjustments attempted by the substrate when compared to the freedom available for shorter substrates. Consequently, the substrate probably is not placed optimally above the active site centre which results in differences in hydrolysis between various substrates and hence the specificity. If the substrate is a segment of another protein as observed in vivo, the polypeptide substrate will have further restriction over the freedom of optimal positioning due to the additional restrictions enforced by the remaining part of the ‘substrate protein’. Hence, this description gives a hint to why the hydrolysis of shorter and longer substrates is different.

Through fluorescence measurements on the aspartic proteinases with specific substrates, it was suggested that conformational mobility of groups in the active site play an important role in the mechanism (Fruton, J. S. Mol. Cell. Biochem. 32, 105-114 (1980)). Similarly, flexible domains and sub-domains with in aspartic proteinase have been identified and implicated in the mechanism (Cele Abad-Zapatero, Timothy J. Rydel, & John Erickson. Proteins: Struc. Funct. Gen. 8, 62-81 (1990), (Andrej Sah, Veerapandian, B., Jon B. Cooper, David S. Moss, Theo Hofmann & Tom L. Blundell. Proteins: Struc. Funct. Gen. 12, 158-170 (1992)). Through this methodology, the most plausible groups or regions which probably play an important role in the catalytic mechanism are more systematically identified.

C-terminal ψ-like Loop Does Not Exist as Claimed Earlier:

There is need to discuss the C-terminal ψ-like loop. Seen from top view, the loop looks like a ψ-like loop. However, in reality, the angle between the loop (217-224) (considering it as a plane) and the strand passing through the loop (297-302) is worked out to be 54°. However, the angle between the central strand of N-terminal ψ-like loop (121-124) and the loop (32-44) is about 9°. This is also evident from the PAC diagrams. Hence, it is likely that the strand does not form part of the ‘CDPL’. There is no C-terminal ψ-like loop comparable to N-terminal ψ-like loop.

Protein as a Tensegrity Structure

On careful examination, tensegrity (Ingber, D. E. Sci. Am. 278, 48-57 (1998)) principles seem to be responsible for the relative PAC mobilities, resulting in the adjustments for optimal positioning and hence efficient catalysis. Tensegrity structures are defined as the interaction of a set of discontinuous (isolated) compression elements (e.g., a PAC) with a set of continuous tension elements (e.g., loops connecting PACs) in the aim to provide a stable volume and shape in the space (Dimitrrje Stamenovic, Jeffrey J. Fredberg, Ning Wang, James P. Butler & Donald E. Ingber. J. Theor. Biol. 181, 125-136 (1996)). The tension elements carry “prestress”, conferring load supporting capability to the entire structure. The role of the compression elements is to provide prestress in tension elements. Together, they form a self-equilibrating, stable mechanical system. The key feature of any tensegrity is the interconnectedness of its elements and the degree of relative motion between the elements making any protein a possible ideal tensegrity structure.

The entry of substrate into the active site probably alters the prestress in the protein molecule. The inherent feature of tensegrity structure to resist deformation enables the relative movement of PACs and the interconnected tensile elements. These relative motions of PACS, in order to balance and redistribute the molecular mechanical stresses, probably provides the force for the intricate movements of different PACs towards the active site centre. Hence, the subtle movements of CDLP, CDWL, FLAP, EXPL, CDPL and NDPL provide proper architectural support, leading to efficient catalysis.

### EXAMPLE 3

Analysis of Retroviral Family:

A similar analysis has been carried out on the retroviral proteinase family also. The retroviral proteinases are evolutionarily related to the pepsin-like family, as indicated by the conserved Asp-Thr(Ser)-Gly sequence and by the overall structural homology, despite the parsimony adopted in their monomer length. The retropepsins are homodimers, with each monomer of about 100 amino acid residues. Each monomer contributes one aspartate (Asp25, HIV numbering) to the active site cleft for catalysis. The geometry of the active site is well conserved across the acid proteinases super family. Interestingly, their specificities show wide variations. This conservation of domain structure across the entire super family of acid proteinases attests to their functional importance. There has been only one report, in which the authors have identified 5 domains in retropepsins which move rigidly relative to one another and have emphasized the functionality of these regions (Robert B. Rose, Charles 5. Craik & Robert M. Stroud. Biochemistry. 37, 2607-2621 (1998)).

Because of the dimeric nature, the components found in retro-viral proteinase family are not as complicated as the pepsin-like family members. This explains the broad specificity of HIV family and very stringent specificity for substrates in pepsin-like proteinases. There is no groove in HIV proteinase for the substrate to travel through, hence no difficulties of chemically unfavorable interactions. The interesting point to note is that the angle between the helix and the equivalent of NDPL and CDPL is still maintained. The angle for NHEL/NDPL equivalent is 15.3° and CHEL/CDPL equivalent is 15.2°. From the preliminary observations of HIV family, it appears that the architectural support for straining the peptide for efficient hydrolysis is different from that of pepsin-like family. The features of the present invention disclosed in the specification, the claims and/or in the accompanying drawings, may, both separately, and in any combination thereof, be material for realising the invention in various forms thereof.

TABLE 1
Aspartic proteinases from different sources whose crystal structures are available in the PDB.

| | | Name of aspartic | | | | | |
|---|---|---|---|---|---|---|---|
| S. No. | PDB Code | proteinase | Resolution(Å) | Number or residues | Sequence identity (%) | RMS Deviation | Stamp score |
| 1 | 2APR | Rhizopuspepsin | 1.8 | 325 | 100.00 | 0.000 | 10.000 |
| 2 | 3APR | Penicillopepsin | 1.8 | 323 | 42.50 | 0.977 | 8.010 |
| 3 | 4APE | Endothiapepsin | 2.2 | 330 | 41.79 | 1.105 | 8.016 |
| 4 | 1MPP | Pusillopepsin | 2.0 | 357 | 31.82 | 1.483 | 7.447 |
| 5 | 2ASI | Mucorpepsin | 2.15 | 356 | 31.75 | 1.483 | 7.647 |
| 6 | 4CMS | Chymosin | 2.2 | 320 | 35.59 | 1.171 | 8.006 |
| 7 | 1AM5 | Atlantic Cod Pepsin | 2.16 | 324 | 35.84 | 0.922 | 7.791 |
| 8 | 1PSN | Human Pepsin | 2.2 | 326 | 40.63 | 1.168 | 8.114 |
| 9 | 4PEP | Porcine Pepsin | 1.8 | 326 | 39.08 | 1.242 | 7.995 |
| 10 | 1BBS | Human Renin | 2.8 | 337 | 27.76 | 1.465 | 7.635 |
| 11 | 1LYA | Cathepsin D | 2.5 | 338 | 33.68 | 1.171 | 7.940 |
| 12 | 1B5F | Cardosin | 1.75 | 320 | 34.04 | 1.201 | 7.906 |

TABLE 3
The angles (°) between a selected set of PACs in all the native aspartic
proteinases. The avarage and standard deviations are also listed.

| | Average | Std.Dev. | 2APR | 3APP | 4APE | 1MPP | 2ASI | 1AM5 |
|---|---|---|---|---|---|---|---|---|
| BASE_ENPL | 28.677 | 19.642 | 9.282 | 11.743 | 17.316 | 69.769 | 68.224 | 27.190 |
| BASE_EXPL | 35.285 | 11.636 | 47.832 | 49.515 | 56.081 | 41.533 | 25.493 | 35.388 |
| BASE_CDPL | 25.169 | 2.387 | 25.794 | 26.657 | 24.327 | 26.625 | 27.667 | 22.905 |
| BASE_NDPL | 37.297 | 2.645 | 36.545 | 38.185 | 36.260 | 34.589 | 35.676 | 39.190 |
| BASE_FLAP | 14.136 | 4.864 | 7.384 | 11.532 | 14.814 | 12.998 | 16.481 | 11.954 |
| NDPL_CDPL | 54.576 | 3.391 | 55.895 | 56.351 | 51.806 | 52.308 | 53.123 | 51.860 |
| NDPL_FLAP | 38.636 | 5.116 | 38.409 | 47.645 | 37.622 | 36.764 | 44.730 | 35.314 |
| NDPL_NHEL | 18.486 | 12.541 | 10.730 | 31.035 | 46.144 | 9.539 | 9.677 | 17.924 |
| CDPL_CDWL | 52.069 | 3.413 | 56.320 | 48.988 | 45.597 | 57.682 | 53.883 | 52.564 |
| CDPL_CHEL | 18.366 | 16.133 | 8.487 | 24.959 | 65.960 | 7.012 | 9.658 | 17.725 |
| CDPL_CDLP | 67.219 | 17.298 | 78.255 | 90.370 | 68.415 | 78.625 | 34.396 | 35.868 |
| CDWL_SBWL | 19.316 | 6.081 | 32.618 | 17.482 | 27.708 | 14.902 | 11.354 | 17.903 |
| CDWL_CDLP | 83.229 | 41.204 | 31.047 | 43.131 | 113.757 | 21.754 | 88.153 | 78.489 |
| SBWL_SBLP | 56.728 | 15.536 | 49.019 | 73.627 | 83.165 | 68.791 | 64.618 | 35.789 |
| NHEL_CHEL | 54.230 | 6.829 | 56.305 | 45.464 | 54.339 | 50.504 | 52.693 | 50.014 |

| | 4CMS | 1PSN | 4PEP | 1BBS | 1LYA | 1B5F |
|---|---|---|---|---|---|---|
| BASE_ENPL | 22.509 | 23.142 | 24.354 | 26.003 | 25.549 | 19.041 |
| BASE_EXPL | 28.032 | 30.080 | 28.012 | 15.171 | 30.333 | 35.946 |
| BASE_CDPL | 28.182 | 24.905 | 24.393 | 21.392 | 21.332 | 27.845 |
| BASE_NDPL | 37.318 | 39.938 | 32.207 | 35.887 | 41.340 | 40.425 |
| BASE_FLAP | 25.820 | 12.606 | 10.458 | 14.793 | 10.931 | 19.858 |
| NDPL_CDPL | 56.731 | 56.204 | 51.775 | 49.904 | 56.799 | 62.153 |
| NDPL_FLAP | 43.051 | 36.254 | 34.244 | 29.713 | 36.308 | 43.579 |
| NDPL_NHEL | 16.648 | 13.957 | 15.220 | 10.916 | 35.719 | 4.318 |
| CDPL_CDWL | 52.042 | 53.513 | 53.162 | 53.135 | 49.083 | 48.861 |
| CDPL_CHEL | 9.199 | 12.012 | 20.120 | 7.376 | 17.754 | 20.126 |
| CDPL_CDLP | 78.068 | 72.181 | 71.131 | 65.960 | 78.557 | 54.802 |
| CDWL_SBWL | 18.392 | 17.814 | 15.290 | 22.954 | 13.616 | 21.772 |
| CDWL_CDLP | 29.412 | 123.528 | 121.980 | 119.092 | 127.625 | 100.779 |
| SBWL_SBLP | 54.274 | 50.005 | 45.574 | 33.235 | 71.196 | 51.447 |
| NHEL_CHEL | 61.033 | 59.838 | 50.703 | 54.068 | 69.748 | 46.055 |

TABLE 4
The normalized B-factors in all the native aspartic proteinases. Average and their standard deviation are also listed.

| | Average | Std.Dev. | 2APR | 3APP | 4APE | 1MPP | 2ASI | 1AM5 | 4CMS | 1PSN | 4PEP | 1BBS | 1LYA | 1B5F |
|---|---|---|---|---|---|---|---|---|---|---|---|---|---|---|
| CDPL | −0.661 | 0.147 | −0.845 | −0.769 | −0.712 | −0.659 | −0.745 | −0.719 | −0.417 | −0.428 | −0.651 | −0.578 | −0.985 | −0.498 |
| BASE | −0.348 | 0.195 | −0.485 | −0.236 | −0.188 | −0.364 | −0.544 | −0.603 | −0.022 | −0.209 | −0.478 | −0.264 | −0.104 | −0.234 |
| NDPL | −0.823 | 0.229 | −0.838 | −0.795 | −0.495 | −0.761 | −1.141 | −1.120 | −0.587 | −0.658 | −1.009 | −0.499 | −0.754 | −0.506 |
| CHEL | −0.626 | 0.257 | −0.854 | −0.738 | −0.220 | −0.330 | −0.777 | −0.951 | −0.361 | −0.718 | −0.688 | −0.790 | −0.884 | −0.743 |
| CDWL | −0.292 | 0.246 | −0.267 | −0.758 | −0.162 | 0.053 | −0.129 | −0.294 | −0.476 | −0.481 | −0.110 | −0.429 | −0.974 | −0.547 |
| NHEL | −0.586 | 0.33 | −0.854 | −0.171 | −0.648 | −0.667 | −0.990 | −0.537 | −0.272 | −0.456 | −0.679 | 0.072 | −0.411 | −0.125 |
| EXPL | −0.249 | 0.232 | −0.271 | −0.417 | −0.010 | 0.143 | −0.520 | −0.386 | −0.352 | −0.425 | −0.006 | 0.503 | −0.911 | −0.455 |
| FLAP | 0.607 | 0.590 | 1.395 | 0.063 | 1.007 | 0.209 | 0.660 | 1.277 | 0.072 | 0.987 | −0.211 | −0.034 | 0.514 | 0.795 |
| SBWL | 0.668 | 0.755 | 1.127 | 0.691 | −0.449 | −0.281 | 0.348 | 0.213 | 1.542 | 1.527 | 1.294 | 0.098 | −0.153 | −0.128 |
| ENPL | 0.092 | 0.803 | 0.039 | 0.655 | −0.276 | −0.485 | −0.748 | −0.289 | 1.946 | 0.165 | −0.178 | −0.664 | −0.114 | −0.436 |
| CDLP | 1.096 | 0.912 | 1.664 | −0.030 | −0.034 | 2.319 | 1.638 | 1.358 | 0.032 | 0.943 | 1.977 | 0.446 | −0.671 | −0.106 |
| SBLP | 2.165 | 1.330 | 2.322 | 4.303 | 0.075 | 0.923 | 1.677 | 1.524 | 3.210 | 1.926 | 3.528 | 1.995 | 2.881 | 1.628 |

1. A method of identifying and/or designing and/or modifying and/or optimizing species capable of interacting with a macromolecule, comprising the steps:
a) defining a set of physiological functions and/or properties of said macromolecule and/or said species, said physiological functions and/or properties being based on empirical data, available for said macromolecule and/or said species,
b) identifying a mechanical analogue from the set of physiological functions and/or properties of said macromolecule and/or said species, which mechanical analogue performs a mechanical function that is analogous to the physiological function of said macromolecule and/or species, and which mechanical analogue performs the mechanical function as a whole or which mechanical analogue consists of parts allowing said mechanical analogue to perform its mechanical function, such that each of said parts of said mechanical analogue performs a component of said mechanical function,
c) providing at least one structure of said macromolecule and/or species, said structure being a representation of the arrangement and connectivity of the atoms of said macromolecule and/or said species, in three dimensional space, or said structure being a set of all coordinates of the atoms of said macromolecule and/or species in three dimensional space, said macromolecule and/or species consisting of building blocks, referred to as residues,
d) identifying a group of residues within the structure of said macromolecule and/or species, said group of residues performing a specific component of physiological function of said macromolecule and/or species, or said group of residues performing a part of said physiological function of said macromolecule and/or species, which group of residues are analogous to a part of said mechanical analogue (identified in b) which performs a part of said mechanical function, said group of residues being referred to as a macromolecule architectural component (MAC), said component of physiological function of said macromolecule having its counterpart in at least one part of the mechanical analogue, identified in b), which mechanical analogue part performs an analogous component of mechanical function in said mechanical analogue,
e) repeating step d) as many times as necessary until all macromolecule architectural components are identified which are necessary for said macromolecule and/or species to perform its physiological function,
f) representing each MAC identified in step d) by a geometrical shape, which shape approximates the dimensions of said MAC,
g) assigning the approximate dimensions to each geometrical shape of step f), thereby defining the coordinates and dimensions of each MAC,
h) calculating the centres of mass and inter-MAC angles using the coordinates of each MAC, and
i) parameterising the identification/design/modification/optimization of species capable of interacting with said macromolecule, by using the inter MAC-angles, centres of mass and the dimensions of the MACs.

2. The method according to claim 1, further comprising the steps of:
j) physically providing/designing/modifying/optimizing a species suspected of interacting with said macromolecule, the identity of said species being based on information retrieved from performing steps a)-i) on said species as well as on said macromolecule,
k) physically providing said macromolecule, and
l) physically mixing said species and said macromolecule and measuring an interaction.

3. The method according to claim 2, wherein the order of steps j) and k) is reversed.

4. The method according to claim 1, wherein said macromolecule is selected from the group consisting of proteins, nucleic acids, carbohydrates, lipids and fats.

5. The method according to claim 4, wherein said macromolecule is selected from the group consisting of A-DNA, B-DNA, Z-DNA, RNA, ribozymes, proteins, protein complexes, peptides, peptidoglycans, carbohydrates, lipids and fats.

6. The method according to claim 1, wherein said species is selected from the group consisting of proteins, peptides, nucleic acids, carbohydrates, lipids, fats, non-protein co-factors, small-molecule-compounds, radicals, ions and macromolecule associated water molecules.

7. The method according to claim 6, wherein said small-molecule-compounds have a molecular mass in the range of 150-1300.

8. The method according to claim 1, wherein the approximate dimensions assigned in step g) are in Ångstroms.

9. The method according to claim 1, wherein said macromolecule is pictorially represented using inter-MAC angles, centres of mass of said MACs and said dimensions of said MACs.

10. The method according to claim 9, wherein the pictorial representation is by means of the geometrical shapes identified in step f), whereby each MAC is represented independently by a geometrical shape, such that the geometrical shape of one MAC may be the same as that of another MAC or they may be different.

11. The method according to claim 1, wherein in the steps d) to f) the MACs are construed by referring to said structure or to said set of coordinates provided in step c), to assign a geometric shape and dimensions, based on said empirical data.

12. The method according to claim 1, wherein the macromolecule architectural component identified in d) comprises residues which are more than 2 Å apart.

13. The method according to claim 1, wherein the macromolecule architectural component occurs within a part of the tertiary structure of the macromolecule that is well defined, as judged by X-ray-data and/or NMR-data and/or homology modeling studies.

14. The method according to claim 13, wherein said macromolecule is a protein.

15. The method according to claim 14, wherein the macromolecule architectural component occurs within a part of the tertiary structure of the protein, the Cα-atoms of which have B-factors in the range of from 2 Å²-200 Å².

16. The method according to claim 13, wherein the macromolecule architectural component occurs in a region of the tertiary structure of the macromolecule the backbone atoms of which have a root mean square deviation (RMSD) in the range of from 0.05 Å-4.0 Å.

17. The method according to claim 1, wherein said macromolecule architectural component(s) is (are independently) represented by a geometrical shape, said shape being selected from the group consisting of planes, parallelepipeds, cubes, cylinders, spirals, rings, tori, ellipsoids, balls and any combination thereof.

18. The method according to claim 17, wherein said geometrical shape represents/is similar to a mechanical part of a machine, such as planks/sheets, springs, tubes, screws, bolts, nuts, rivets, bushings, bearings and other components used for manufacturing a machine or component of a machine.

19. The method according to claim 17, wherein the geometrical shape selected for a MAC is a plane.

20. The method according to claim 19, wherein a macromolecule architectural component is represented by a difference vector matrix A, wherein
‘A’=[(xᵢ−<x>) (yᵢ−<y>) (zᵢ−<z>)]
(xᵢ, yᵢ, zᵢ)=[X, Y, Z] coordinates of the central atom of each residue in the MAC, e.g. of the Cα atom, in the PAC
(<x>, <y>, <z>)=(Σxᵢ/n, Σyᵢ/n, Σzᵢ/n)
n=Number of central atoms of each residue in each MAC, e.g. of Cα atoms in each PAC.

21. The method according to claim 20, wherein said difference vector matrix A is solved to yield a singular vector which represents the direction cosine of the vector which is normal to the best-fitting plane of the given coordinates of the macromolecule architectural component.

22. The method according to claim 17, wherein the geometrical shape selected for a MAC is a cylinder or a spiral.

23. The method according to claim 22, wherein a directional vector intersecting with the longitudinal axis of said cylinder or spiral is calculated.

24. The method according to claim 23, wherein said calculation occurs by the method of besection of vectors.

25. The method according to claim 1, wherein the physiological functions and/or properties of the macromolecule are selected from the group consisting of oxidoreductase, transferase, hydrolase, lyase, isomerase and ligase.

26. The method according to claim 25, wherein the physiological functions and/or properties of the macromolecule are selected from the group consisting of protease, kinase, phosphorylase, DNAase, RNAase, lipase and polymerase.

27. The method according to claim 1, wherein the physiological functions and/or properties of the macromolecule are selected from the group consisting of regulatory function in cell metabolism, regulatory function in transcription and/or translation, regulatory function in signal transduction pathway, structural function, storage function, motility function, transport function, and recognition function.

28. The method according to claim 2, wherein the measurement of an interaction between the species suspected of interacting with said macromolecule and said macromolecule, in step 1), occurs by UV-vis-absorption spectroscopy, fluorescence spectroscopy, circular dichroism, NMR-spectroscopy, surface plasmon resonance spectroscopy, gelfiltration, ultracentrifugation, viscometry, electrophoresis, and/or any combination of the aforementioned techniques.

29. A species and/or macromolecule identified/designed/modified/optimized by a method of identifying and/or modifying and/or optimizing species capable of interacting with a macromolecule, comprising the steps:
a) defining a set of physiological functions and/or properties of said macromolecule and/or said species, said physiological functions and/or properties being based on empirical data, available for said macromolecule and/or said species,
b) identifying a mechanical analogue from the set of physiological functions and/or properties of said macromolecule and/or said species, which mechanical analogue performs a mechanical function that is analogous to the physiological function of said macromolecule and/or species, and which mechanical analogue performs the mechanical function as a whole or which mechanical analogue consists of parts allowing said mechanical analogue to perform its mechanical function, such that each of said parts of said mechanical analogue performs a component of said mechanical function,
c) providing at least one structure of said macromolecule and/or species, said structure being a representation of the arrangement and connectivity of the atoms of said macromolecule and/or said species, in three dimensional space, or said structure being a set of all coordinates of the atoms of said macromolecule and/or species in three dimensional space, said macromolecule and/or species consisting of building blocks, referred to as residues,
d) identifying a group of residues within the structure of said macromolecule and/or species, said group of residues performing a specific component of physiological function of said macromolecule and/or species, or said group of residues performing a part of said physiological function of said macromolecule and/or species, which group of residues are analogous to a part of said mechanical analogue (identified in b) which performs a part of said mechanical function, said group of residues being referred to as a macromolecule architectural component (MAC), said component of physiological function of said macromolecule having its counterpart in at least one part of the mechanical analogue, identified in b), which mechanical analogue part performs an analogous component of mechanical function in said mechanical analogue,
e) repeating step d) as many times as necessary until all macromolecule architectural components are identified which are necessary for said macromolecule and/or species to perform its physiological function,
f) representing each MAC identified in step d) by a geometrical shape, which shape approximates the dimensions of said MAC,
g) assigning the approximate dimensions to each geometrical shape of step f), thereby defining the coordinates and dimensions of each MAC,
h) calculating the centres of mass and inter-MAC angles using the coordinates of each MAC, and
i) parameterising the identification/design/modification/optimization of species capable of interacting with said macromolecule, by using the inter MAC-angles, centres of mass and the dimensions of the MACs.

30. A graphical representation of a species and/or macromolecule identified/designed/modified/optimized by a method of identifying and/or modifying and/or optimizing species capable of interacting with a macromolecule, comprising the steps:
a) defining a set of physiological functions and/or properties of said macromolecule and/or said species, said physiological functions and/or properties being based on empirical data, available for said macromolecule and/or said species,
b) identifying a mechanical analogue from the set of physiological functions and/or properties of said macromolecule and/or said species, which mechanical analogue performs a mechanical function that is analogous to the physiological function of said macromolecule and/or species, and which mechanical analogue performs the mechanical function as a whole or which mechanical analogue consists of parts allowing said mechanical analogue to perform its mechanical function, such that each of said parts of said mechanical analogue performs a component of said mechanical function,
c) providing at least one structure of said macromolecule and/or species, said structure being a representation of the arrangement and connectivity of the atoms of said macromolecule and/or said species, in three dimensional space, or said structure being a set of all coordinates of the atoms of said macromolecule and/or species in three dimensional space, said macromolecule and/or species consisting of building blocks, referred to as residues,
d) identifying a group of residues within the structure of said macromolecule and/or species, said group of residues performing a specific component of physiological function of said macromolecule and/or species, or said group of residues performing a part of said physiological function of said macromolecule and/or species, which group of residues are analogous to a part of said mechanical analogue (identified in b) which performs a part of said mechanical function, said group of residues being referred to as a macromolecule architectural component (MAC), said component of physiological function of said macromolecule having its counterpart in at least one part of the mechanical analogue, identified in b), which mechanical analogue part performs an analogous component of mechanical function in said mechanical analogue,
e) repeating step d) as many times as necessary until all macromolecule architectural components are identified which are necessary for said macromolecule and/or species to perform its physiological function,
f) representing each MAC identified in step d) by a geometrical shape, which shape approximates the dimensions of said MAC,
g) assigning the approximate dimensions to each geometrical shape of step f), thereby defining the coordinates of each MAC,
h) calculating the centres of mass and inter-MAC angles using the coordinates of each MAC, and
i) parameterising the identification/design/modification/optimization of species capable of interacting with said macromolecule, by using the inter MAC-angles, centres of mass and the dimensions of the MACs.
